SEQUENCE LISTING (90 sequences)

SEQ ID NO: 1 - protein, 275 aa, Artificial Sequence
  Bacillus subtilis flagellin protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLR

SEQ ID NO: 2 - protein, 505 aa, Artificial Sequence
  Salmonella dublin flagellin protein
MAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNGPKEATVGDLKSSFKNVTGYDTYAAGADKYRVDINSGAVVTDAVAPDKVYVNAANGQLTTDDAENNTAVDLFKTTKSTAGTAEAKAIAGAIKGGKEGDTFDYKGVTFTIDTKTGDDGNGKVSTTINGEKVTLTVADIAIGAADVNAATLQSSKNVYTSVVNGQFTFDDKTKNESAKLSDLEANNAVKGESKITVNGAEYTANATGDKITLAGKTMFIDKTASGVSTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 3 - protein, 387 aa, Artificial Sequence
  Pseudomonas aeruginosa flagellin protein
MALTVNTNIASLNTQRNQNNSSASLNTSLQRLSTGSRINSAKDDAAGLQIANRLTSQVNGLNVATKNANDGISLAQTAEGALQQSTNILQRMRDLSLQSANGSNSDSERTALNGEVKQLQKELDRISNTTTFGGRKLLDGSFGVASFQVGSAANEIISVGIDEMSAESLNGTYFKADGGGAVTAATASGTVDIAIDITGGSAVNVKVDMKGNETAEQAAAKIAAAVNDANVGIGAFTDGAQISYVSKASADGTTSAVSGVAITDTGSTGAGTAAGTTTFTEANDTVAKIDISTAKGAQSAVLVIDEAIKQIDAQRADLGAVQNRFDNTINNLKNIGENVSAARGRIEDTDFAAETANLTKNQVLQQAGTAILAQANQLPQSVLSLLR

SEQ ID NO: 4 - protein, 550 aa, Artificial Sequence
  Shigella flexneri flagellin protein
MAQVINTNSLSLITQNNINKNQSALSSSIERLSSGLRINSAKDDAAGQAIANRFTSNIKGLTQAARNANDGISVAQTTEGALSEINNNLQRIRELTVQASTGTNSDSDLDSIQDEIKSRLDEIDRVSGQTQFNGVNVLAKDGSMKIQVGANDGQTITIDLKKIDSDTLGLNGFNVNGGGAVANTAASKADLVAANATVVGNKYTVSAGYDAAKASDLLAGVSDGDTVQATINNGFGTAASATNYKYDSASKSYSFDTTTASAADVQKYLTPGVGDTAKGTITIDGSAQDVQISSDGKITASNGDKLYIDTTGRLTKNGSGASLTEASLSTLAANNTKATTIDIGGTSISFTGNSTTPDTITYSVTGAKVDQAAFDKAVSTSGNNVDFTTAGYSVNGTTGAVTKGVDSVYVDNNEALTTSDTVDFYLQDDGSVTNGSGKAVYKDADGKLTTDAETKAATTADPLKALDEAISSIDKFRSSLGAVQNRLDSAVTNLNNTTTNLSEAQSRIQDADYATEVSNMSKAQIIQQAGNSVLAKANQVPQQVLSLLQG

SEQ ID NO: 5 - protein, 498 aa, Artificial Sequence
  Escherichia coli flagellin protein
MAQVINTNSLSLITQNNINKNQSALSSSIERLSSGLRINSAKDDAAGQAIANRFTSNIKGLTQAARNANDGISVAQTTEGALSEINNNLQRVRELTVQATTGTNSESDLSSIQDEIKSRLDEIDRVSGQTQFNGVNVLAKNGSMKIQVGANDNQTITIDLKQIDAKTLGLDGFSVKNNDTVTTSAPVTAFGATTTNNIKLTGITLSTEAATDTGGTNPASIEGVYTDNGNDYYAKITGGDNDGKYYAVTVANDGTVTMATGATANATVTDANTTKATTITSGGTPVQIDNTAGSATANLGAVSLVKLQDSKGNDTDTYALKDTNGNLYAADVNETTGAVSVKTITYTDSSGAASSPTAVKLGGDDGKTEVVDIDGKTYDSADLNGGNLQTGLTAGGEALTAVANGKTTDPLKALDDAIASVDKFRSSLGAVQNRLDSAVTNLNNTTTNLSEAQSRIQDADYATEVSNMSKAQIIQQAGNSVLAKANQVPQQVLSLLQG

SEQ ID NO: 6 - protein, 229 aa, Artificial Sequence
  hIgG4 Fc S228P mutant - PGK
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 7 - protein, 229 aa, Artificial Sequence
  hIgG4 Fc S228P mutant + S220P (PGK)
EPKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 8 - protein, 229 aa, Artificial Sequence
  hIgG4 Fc S228P mutant + G223T (PGK)
ESKYTPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 9 - protein, 229 aa, Artificial Sequence
  hIgG4 Fc S228P mutant + P224H (PGK)
ESKYGHPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 10 - protein, 229 aa, Artificial Sequence
  hIgG4 Fc S228P mutant + P225T
ESKYGPTCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 11 - protein, 229 aa, Artificial Sequence
  hIgG4 Fc S228P mutant - LGK
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 5 aa, Artificial Sequence
  Linker 1
GGGGS

SEQ ID NO: 13 - protein, 15 aa, Artificial Sequence
  Linker 2
GGGGSGGGGSGGGGS

SEQ ID NO: 14 - DNA, 43 bp, Artificial Sequence
  pFUSE-hIgG4-fc2-bsFlagellin-partial hinge Forward primer
ccggatatcgatgagaattaaccacaatattgcagcacttaac

SEQ ID NO: 15 - DNA, 54 bp, Artificial Sequence
  pFUSE-hIgG4-fc2-bsFlagellin-partial hinge Reverse primer
gaccatggcagaccctccgccaccacgtaataattgaagtacgttttgaggctg

SEQ ID NO: 16 - DNA, 43 bp, Artificial Sequence; identical to SEQ ID NO: 14
  pFUSE-hIgG4-fc2-bsFlagellin-partial hinge Forward primer

SEQ ID NO: 17 - DNA, 84 bp, Artificial Sequence
  pFUSE-hIgG4-fc2-bsFlagellin-partial hinge Reverse primer
gaccatggcagaccctccgccaccagaccctccgccaccagaccctccgccaccacgtaataattgaagtacgttttgaggctg

SEQ ID NO: 18 - DNA, 43 bp, Artificial Sequence; identical to SEQ ID NO: 14
  pFUSE-hIgG4-fc2-bsFlagellin-partial hinge Forward primer

SEQ ID NO: 19 - DNA, 32 bp, Artificial Sequence
  pFUSE-hIgG4-fc2-bsFlagellin-partial hinge Reverse primer
tcagatctaaccatggcacgtaataattgaag

SEQ ID NO: 20 - DNA, 48 bp, Artificial Sequence
  pFUSE-hIgG4-fc2-bsFlagellin-full hinge-CPSC Forward primer
tatatccatggttagatctgaatccaaatacggtcccccatgcccatc

SEQ ID NO: 21 - DNA, 37 bp, Artificial Sequence
  pFUSE-hIgG4-fc2-bsFlagellin-full hinge-CPSC Reverse primer
tatatgctagcactcatttacccagagacagggagag

SEQ ID NO: 22 - DNA, 43 bp, Artificial Sequence; identical to SEQ ID NO: 14
  pFUSE-hIgG4-fc2-bsFlagellin-full hinge-CPSC Forward primer

SEQ ID NO: 23 - DNA, 32 bp, Artificial Sequence; identical to SEQ ID NO: 19
  pFUSE-hIgG4-fc2-bsFlagellin-full hinge-CPSC Reverse primer

SEQ ID NO: 24 - DNA, 63 bp, Artificial Sequence
  pFUSE-hIgG4-fc2-bsFlagellin-full hinge Forward primer
tatatccatggttagatctgaatccaaatacggtcccccatgcccaccttgcccagcacctga

SEQ ID NO: 25 - DNA, 37 bp, Artificial Sequence; identical to SEQ ID NO: 21
  pFUSE-hIgG4-fc2-bsFlagellin-full hinge Reverse primer

SEQ ID NO: 26 - DNA, 43 bp, Artificial Sequence; identical to SEQ ID NO: 14
  pFUSE-hIgG4-fc2-bsFlagellin-full hinge-CPPC Forward primer

SEQ ID NO: 27 - DNA, 32 bp, Artificial Sequence; identical to SEQ ID NO: 19
  pFUSE-hIgG4-fc2-bsFlagellin-full hinge-CPPC Reverse primer

SEQ ID NO: 28 - DNA, 57 bp, Artificial Sequence
  pFUSE-hIgG4-fc2-bsFlagellin-full hinge-CPPC-S220P Forward primer
ggagggtctgccatggttagatctgaacccaaatacggtcccccatgcccaccttgc

SEQ ID NO: 29 - DNA, 37 bp, Artificial Sequence; identical to SEQ ID NO: 21
  pFUSE-hIgG4-fc2-bsFlagellin-full hinge-CPPC-S220P Reverse primer

SEQ ID NO: 30 - DNA, 57 bp, Artificial Sequence
  pFUSE-hIgG4-fc2-bsFlagellin-full hinge-CPPC-G223T Forward primer
ggagggtctgccatggttagatctgaatccaaatacactcccccatgcccaccttgc

SEQ ID NO: 31 - DNA, 37 bp, Artificial Sequence; identical to SEQ ID NO: 21
  pFUSE-hIgG4-fc2-bsFlagellin-full hinge-CPPC-G223T Reverse primer

SEQ ID NO: 32 - DNA, 57 bp, Artificial Sequence
  pFUSE-hIgG4-fc2-bsFlagellin-full hinge-CPPC-P224H Forward primer
ggagggtctgccatggttagatctgaatccaaatacggtcacccatgcccaccttgc

SEQ ID NO: 33 - DNA, 37 bp, Artificial Sequence; identical to SEQ ID NO: 21
  pFUSE-hIgG4-fc2-bsFlagellin-full hinge-CPPC-P224H Reverse primer

SEQ ID NO: 34 - DNA, 57 bp, Artificial Sequence
  pFUSE-hIgG4-fc2-bsFlagellin-full hinge-CPPC-P225T Forward primer
ggagggtctgccatggttagatctgaatccaaatacggtcccacatgcccaccttgc

SEQ ID NO: 35 - DNA, 37 bp, Artificial Sequence; identical to SEQ ID NO: 21
  pFUSE-hIgG4-fc2-bsFlagellin-full hinge-CPPC-P225T Reverse primer

SEQ ID NO: 36 - DNA, 48 bp, Artificial Sequence
  pFUSE-hIgG4-fc2-bsFlagellin-full hinge-CPPC (MSP306L) Forward primer
gacaaggatatcgatgagaattaaccacaatattgcagcacttaacac

SEQ ID NO: 37 - DNA, 35 bp, Artificial Sequence
  pFUSE-hIgG4-fc2-bsFlagellin-full hinge-CPPC (MSP306L) Reverse primer
gacgctagctcatttacccagagacagggagaggc

SEQ ID NO: 38 - DNA, 1530 bp, Artificial Sequence
  pFUSE-hIgG4-fc2-bsFlagellin-partial hinge (MSP303) DNA
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtggtggcggagggtctgccatggttagatctcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 39 - protein, 509 aa, Artificial Sequence
  pFUSE-hIgG4-fc2-bsFlagellin-partial hinge (MSP303) Protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 40 - DNA, 1560 bp, Artificial Sequence
  pFUSE-hIgG4-fc2-bsFlagellin-partial hinge (MSP304) DNA
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtggtggcggagggtctggtggcggagggtctggtggcggagggtctgccatggttagatctcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 41 - protein, 519 aa, Artificial Sequence
  pFUSE-hIgG4-fc2-bsFlagellin-partial hinge (MSP304) Protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSGGGGSGGGGSAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 42 - DNA, 1515 bp, Artificial Sequence
  pFUSE-hIgG4-fc2-bsFlagellin-partial hinge-NL DNA
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtgccatggttagatctcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 43 - protein, 504 aa, Artificial Sequence
  pFUSE-hIgG4-fc2-bsFlagellin-partial hinge-NL Protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 44 - DNA, 1545 bp, Artificial Sequence
  pFUSE-hIgG4-fc2-bsFlagellin-full hinge-CPSC (MSP305) DNA
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtggtggcggagggtctgccatggttagatctgaatccaaatacggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 45 - protein, 514 aa, Artificial Sequence
  pFUSE-hIgG4-fc2-bsFlagellin-full hinge-CPSC (MSP305) Protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSAMVRSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 46 - DNA, 1530 bp, Artificial Sequence
  pFUSE-hIgG4-fc2-bsFlagellin-full hinge-CPSC-NL DNA
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtgccatggttagatctgaatccaaatacggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccccgtctccgggtaaatga

SEQ ID NO: 47 - protein, 509 aa, Artificial Sequence
  pFUSE-hIgG4-fc2-bsFlagellin-full hinge-CPSC-NL Protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRAMVRSESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSPSPGK

SEQ ID NO: 48 - DNA, 1545 bp, Artificial Sequence
  pFUSE-hIgG4-fc2-bsFlagellin-full hinge-CPPC (MSP306) DNA
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtggtggcggagggtctgccatggttagatctgaatccaaatacggtcccccatgcccaccttgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 49 - protein, 514 aa, Artificial Sequence
  pFUSE-hIgG4-fc2-bsFlagellin-full hinge-CPPC (MSP306) Protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSAMVRSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 50 - DNA, 1530 bp, Artificial Sequence
  pFUSE-hIgG4-fc2-bsFlagellin-full hinge-CPPC-NL DNA
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtgccatggttagatctgaatccaaatacggtcccccatgcccaccttgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 51 - protein, 509 aa, Artificial Sequence
  pFUSE-hIgG4-fc2-bsFlagellin-full hinge-CPPC-NL Protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRAMVRSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 52 - DNA, 1545 bp, Artificial Sequence
  pFUSE-hIgG4-fc2-bsFlagellin-full hinge-CPPC-S220P DNA
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtggtggcggagggtctgccatggttagatctgaacccaaatacggtcccccatgcccaccttgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 53 - protein, 514 aa, Artificial Sequence
  pFUSE-hIgG4-fc2-bsFlagellin-full hinge-CPPC-S220P Protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSAMVRSEPKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 54 - DNA, 1545 bp, Artificial Sequence
  pFUSE-hIgG4-fc2-bsFlagellin-full hinge-CPPC-G223T DNA
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtggtggcggagggtctgccatggttagatctgaatccaaatacactcccccatgcccaccttgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 55 - protein, 514 aa, Artificial Sequence
  pFUSE-hIgG4-fc2-bsFlagellin-full hinge-CPPC-G223T Protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSAMVRSESKYTPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 56 - DNA, 1545 bp, Artificial Sequence
  pFUSE-hIgG4-fc2-bsFlagellin-full hinge-CPPC-P224H DNA
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtggtggcggagggtctgccatggttagatctgaatccaaatacggtcacccatgcccaccttgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 57 - protein, 514 aa, Artificial Sequence
  pFUSE-hIgG4-fc2-bsFlagellin-full hinge-CPPC-P224H Protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSAMVRSESKYGHPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 58 - DNA, 1545 bp, Artificial Sequence
  pFUSE-hIgG4-fc2-bsFlagellin-full hinge-CPPC-P225T DNA
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtggtggcggagggtctgccatggttagatctgaatccaaatacggtcccacatgcccaccttgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 59 - protein, 514 aa, Artificial Sequence
  pFUSE-hIgG4-fc2-bsFlagellin-full hinge-CPPC-P225T Protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSAMVRSESKYGPTCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 60 - DNA, 1545 bp, Artificial Sequence
  pFUSE-hIgG4-fc2-bsFlagellin-full hinge-CPPC (MSP306L) DNA
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtggtggcggagggtctgccatggttagatctgaatccaaatacggtcccccatgcccaccttgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 61 - protein, 514 aa, Artificial Sequence
  pFUSE-hIgG4-fc2-bsFlagellin-full hinge-CPPC (MSP306L) Protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSAMVRSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - DNA, 1590 bp, Artificial Sequence
  pAcGP67-A-bsFlagellin-full hinge-CPPC (MSP306L-Insect) DNA
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtggtggcggagggtctgccatggttagatctgaatccaaatacggtcccccatgcccaccttgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgcggccgcctggttccgcgtggttcgcatcatcatcatcatcactga

SEQ ID NO: 63 - protein, 529 aa, Artificial Sequence
  pAcGP67-A-bsFlagellin-full hinge-CPPC (MSP306L-Insect) Protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSAMVRSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKCGRLVPRGSHHHHHH

SEQ ID NO: 64 - protein, 2 aa, Artificial Sequence
  peptide linker 1
GG

SEQ ID NO: 65 - protein, 3 aa, Artificial Sequence
  peptide linker 2
GAG

SEQ ID NO: 66 - protein, 3 aa, Artificial Sequence
  peptide linker 3
GPA

SEQ ID NO: 67 - protein, 1 aa, Artificial Sequence
  peptide linker 4
G

SEQ ID NO: 68 - protein, 2 aa, Artificial Sequence
  peptide linker 5
GS

SEQ ID NO: 69 - protein, 5 aa, Artificial Sequence
  peptide linker 6
GSGSG

SEQ ID NO: 70 - protein, 3 aa, Artificial Sequence
  peptide linker 7
GGS

SEQ ID NO: 71 - protein, 5 aa, Artificial Sequence
  peptide linker 8
GGSSG

SEQ ID NO: 72 - protein, 4 aa, Artificial Sequence
  peptide linker 9
GGSG

SEQ ID NO: 73 - protein, 4 aa, Artificial Sequence
  peptide linker 10
GGGS

SEQ ID NO: 74 - protein, 6 aa, Artificial Sequence
  peptide linker 11
GGSGGG

SEQ ID NO: 75 - protein, 13 aa, Artificial Sequence
  peptide linker 12
GSGGGGSGGGGSG

SEQ ID NO: 76 - protein, 12 aa, Artificial Sequence
  IgG4 Fc hinge 1
ESKYGPPCPSCP

SEQ ID NO: 77 - protein, 12 aa, Artificial Sequence
  IgG4 Fc hinge 2
ESKYGPPCPPCP

SEQ ID NO: 78 - protein, 11 aa, Artificial Sequence
  IgG4 Fc hinge 3
SKYGPPCPPCP

SEQ ID NO: 79 - protein, 10 aa, Artificial Sequence
  IgG4 Fc hinge 4
KYGPPCPPCP

SEQ ID NO: 80 - protein, 9 aa, Artificial Sequence
  IgG4 Fc hinge 5
YGPPCPPCP

SEQ ID NO: 81 - protein, 8 aa, Artificial Sequence
  IgG4 Fc hinge 6
GPPCPPCP

SEQ ID NO: 82 - protein, 7 aa, Artificial Sequence
  IgG4 Fc hinge 7
PPCPPCP

SEQ ID NO: 83 - protein, 6 aa, Artificial Sequence
  IgG4 Fc hinge 8
PCPPCP

SEQ ID NO: 84 - protein, 5 aa, Artificial Sequence
  IgG4 Fc hinge 9
CPPCP

SEQ ID NO: 85 - protein, 4 aa, Artificial Sequence
  IgG4 Fc hinge 10
CPPC

SEQ ID NO: 86 - protein, 7 aa, Artificial Sequence
  IgG4 Fc hinge mutant 1
PPCPSCP

SEQ ID NO: 87 - protein, 12 aa, Artificial Sequence
  IgG4 Fc hinge mutant 2
EPKYGPPCPPCP

SEQ ID NO: 88 - protein, 12 aa, Artificial Sequence
  IgG4 Fc hinge mutant 3
ESKYTPPCPPCP

SEQ ID NO: 89 - protein, 12 aa, Artificial Sequence
  IgG4 Fc hinge mutant 4
ESKYGHPCPPCP

SEQ ID NO: 90 - protein, 12 aa, Artificial Sequence
  IgG4 Fc hinge mutant 5
ESKYGPTCPPCP

(19) United States
(12) Patent Application Publication
(10) Pub. No.: US 2025/0263450 A1
(43) Pub. Date: Aug. 21, 2025

(54) FLAGELLIN FUSION PROTEIN AND USE THEREOF

(71) Applicants: MEDISPAN CO., LTD, Gyeonggi-do (KR); INDUSTRY FOUNDATION OF CHONNAM NATIONAL UNIVERSITY, Gwangju (KR)

(72) Inventor: Kyung A. CHO, Gwangju (KR)

(21) Appl. No.: 18/032,909

(22) PCT Filed: Oct. 20, 2021
(86) PCT No.: PCT/KR2021/014757

§ 371 Date: Apr. 20, 2023

(30) Foreign Application Priority Data
Oct. 20, 2020 (KR) 10-2020-0136273

(51) Int. Cl. C07K 14/32; A61K 38/00

(52) U.S. Cl. CPC C07K 14/32; A61K 38/00; C07K 2319/30

(57) ABSTRACT

The present invention relates to a flagellin fusion protein and the use thereof, and, more specifically, to a fusion protein comprising a human IgG4 Fc variant and the use thereof using toll-like receptor 5 (TLR5) stimulation activity, wherein the human IgG4 Fc variant has mutation preventing a Fab-arm exchange.

This application is a National Phase Application of PCT/KR2021/014757, filed Oct. 20, 2021, which claims priority to Korean Patent Application No. 10-2020-0136273 filed on Oct. 20, 2020, and the entire disclosures of which are incorporated herein by reference.

### REFERENCE TO A SEQUENCE LISTING

A Sequence Listing conforming to the rules of WIPO Standard ST.25 is hereby incorporated by reference. Said Sequence Listing has been filed as an electronic document via PatentCenter in ASCII formatted text. The electronic document, created on Oct. 23, 2023, is entitled “11239-017US1_ST25”, and is 121,590 bytes in size.

### TECHNICAL FIELD

The present invention relates to a flagellin fusion protein and its use. More specifically, the invention relates to a fusion protein comprising a variant of human IgG4 Fc, which has a mutation that prevents Fab arm exchange, and its use Toll-like receptor 5 (TLR5) activation.

### TECHNICAL BACKGROUND OF THE INVENTION

Flagella are important components that determine the motility of bacteria and are composed of a hook, basal body, and filament. Flagella determine bacterial swimming or swarming motility, bacterial taxis, and are known to form biofilms that determine the adhesion ability of pathogenic microorganisms. The protein units that make up the flagella filament are called flagellin, which assemble to form the filament. Hayashi et al. reported that Toll-like receptor 5 (TLR5) expressed in mammals recognizes flagellin of both gram-negative and gram-positive bacteria, activating NF-κB (Hayashi F, Smith K D, Ozinsky A, Hawn T R, Yi E C, Goodlett D R, Eng J K, Akira S, Underhill D M, Aderem A: Nature 410:1099-1103, 2001).

Flagellin is a structural protein that assembles into whip-like filaments of bacterial flagella and functions to enable bacteria to move by extending from the cell surface. As a virulence factor, flagellin acts to facilitate the invasion and intracellular penetration of pathogenic bacteria into host cells. Flagellin is exclusively found in bacteria and is one of the most abundant proteins in flagellated bacteria, making it a major target of host immune surveillance. Upon bacterial invasion, flagellin is detected by Toll-like receptor 5 (TLR5) and NAIP5/NLRC4 in the host, activating innate immunity that contributes to the immediate clearance of pathogens in the host.

TLR5 is a surface-bound innate immune receptor that consists of an extracellular leucine-rich repeat (LRR) domain, a transmembrane domain, and an intracellular domain. TLR5 recognizes flagellin as a pathogen-associated molecular pattern using its extracellular domain and activates MyD88-dependent signaling pathways and NF-κB-mediated inflammatory cytokine production.

Because flagellin plays a critical role as the first line of defense against flagellated-pathogenic bacteria, it has been of interest as a target for vaccine antigen proteins or adjuvants development. Fusion proteins of antigens and flagellin have been shown to be effective as experimental vaccines against various infectious diseases, including West Nile fever, malaria, and tuberculosis. Activation of TLR5 by flagellin has also been reported to protect hematopoietic cells and intestinal tissues from radiation and to affect the survival and growth of cancer cells.

Flagellin contains 2 to 4 domains. For example, Bacillus subtilis Hag flagellin, Pseudomonas aeruginosa A-type FliC flagellin, and Salmonella enterica subspecies enterica serovar Typhimurium FliC flagellin each contain 2 (DO and D1), 3 (DO, D1, and D2), and 4 (DO, D1, D2, and D3) domains, respectively. The common DO and D1 domains mediate inter-flagellar interactions and are located at the center of the flagellar filament. They are highly conserved among bacterial species due to their functional importance in filament formation. Since flagellin monomers, rather than polymerized filaments, activate TLR5, the DO and D1 domains are thought to be the major stimulants of TLR5. In 3 and 4-domain flagellins, the D1 domain extends to the surface of the flagellar filament as an accessory domain (D2 and D3) and contributes almost nothing to filament formation. Unlike the DO and D1 domains, the D2 or D3 domains show significant changes in sequence and structure, activate adaptive immunity, and are considered undesirable for flagellin-based therapy because of their toxicity. Therefore, the radiation therapy biologic drug CBLB502, which contains DO/D1, was developed by removing the hypervariable region (D2 and D3 domains) from Salmonella flagellin.

Many gram-positive bacteria, such as Bacillus subtilis and Clostridium difficile, express flagellin with a lack of hypervariable region, which includes the minimal region (DO and D1 domains) required for TLR5 activation and flagellin polymerization.

The interaction between flagellin and TLR5 and its cellular consequences have been extensively studied using Salmonella flagellin. Structural and biochemical studies of the complex between Salmonella enterica subspecies enterica serovar Dublin flagellin D1-D2 region (sdflagellin D1-D2) and a fragment of zebrafish TLR5's N-terminal reveal that flagellin and TLR5 form a 1:1 complex through ‘primary binding’, followed by homodimerization into a 2:2 complex through ‘secondary oligomerization’.

Based on numerous studies on the interaction between flagellin and TLR5, various studies to enhance TLR5 activation through modification of flagellin have been conducted. However, research on a new form of flagellin protein targeting the flagellin-TLR5 2:2 complex structure is currently lacking.

### INVENTION DESCRIPTION

### Problem to be Solved

The present inventors have repeatedly conducted research to develop a new form of protein that enhances TLR5 activation ability by focusing on the fact that a 2:2 complex structure is formed when flagellin activates TLR5. As a result, they discovered that a new fusion protein in which flagellin and an immunoglobulin Fc were fused showed significantly superior TLR5 activation ability compared to wild-type flagellin, announced flagellin fragments, and the like.

In particular, when producing a flagellin fusion protein using the Fc of human IgG4 to avoid antibody-dependent cell-mediated cytotoxicity (ADCC) or complement-dependent cytotoxicity (CDC) induced by immunoglobulin Fc, it was confirmed that a ½ form of antibody molecule is formed due to Fab arm exchange, which significantly reduces the TLR5 activation ability of flagellin. The present invention was completed by confirming that this problem can be solved by using a mutant of human IgG4 Fc.

Therefore, an object of the present invention is to provide a fusion protein comprising flagellin, fragment, or variant thereof, and a human IgG4 Fc variant, wherein the human IgG4 Fc variant has a mutation that prevents Fab arm exchange.

Another object of the present invention is to provide a polynucleotide encoding the fusion protein.

Another object of the present invention is to provide a vector comprising the polynucleotide.

Another object of the present invention is to provide a transfectant transformed with the vector.

Another object of the present invention is to provide a pharmaceutical composition comprising the fusion protein as an active ingredient.

In addition, another object of the present invention is to provide a pharmaceutical composition consisting of the fusion protein.

In addition, another object of the present invention is to provide a pharmaceutical composition essentially consisting of the fusion protein.

Another object of the present invention is to provide a vaccine adjuvant comprising the fusion protein as an active ingredient.

Another object of the present invention is to provide a vaccine adjuvant consisting of the fusion protein as an active ingredient.

Another object of the present invention is to provide a vaccine adjuvant essentially consisting of the fusion protein as an active ingredient.

Another object of the present invention is to provide a use of the fusion protein for the preparation of an agent for treating radiation exposure-induced damage, reperfusion injury, inflammatory bowel disease, autoimmune disease, viral infection, metabolic disease or cancer, or enhancing immune function.

Another object of the present invention is to provide a method for treating radiation exposure-induced damage, reperfusion injury, inflammatory bowel disease, autoimmune disease, viral infection, metabolic disease or cancer, or enhancing immune function, the method comprising administering an effective amount of a composition comprising the fusion protein as an active ingredient to a subject in need thereof.

### Means for Solving the Problem

In order to the object of the present invention, the present invention provides a fusion protein comprising flagellin, fragment or variant thereof, and a human IgG4 Fc variant, wherein the human IgG4 Fc variant has a mutation that prevents Fab arm exchange.

In order to achieve another object of the present invention, the present invention provides a polynucleotide encoding the fusion protein.

In order to achieve another object of the present invention, the present invention provides a vector comprising the polynucleotide.

In order to achieve another object of the present invention, the present invention provides a transfectant transformed with the vector.

In order to achieve another object of the present invention, the present invention provides a pharmaceutical composition comprising the fusion protein as an active ingredient: In addition, the present invention provides a pharmaceutical composition consisting of the fusion protein.

In addition, the present invention provides a pharmaceutical composition essentially consisting of the fusion protein.

In order to achieve another object of the present invention, the invention provides a vaccine adjuvant comprising the fusion protein as an active ingredient.

In addition, the invention provides a vaccine adjuvant consisting of the fusion protein as an active ingredient.

In addition, the invention provides a vaccine adjuvant essentially consisting of the fusion protein as an active ingredient.

In order to achieve another object of the present invention, the invention provides a use of the fusion protein for the preparation of an agent for treating radiation exposure-induced damage, reperfusion injury, inflammatory bowel disease, autoimmune disease, viral infection, metabolic disease or cancer, or enhancing immune function.

In order to achieve another object of the present invention, the invention provides a method for treating radiation exposure-induced damage, reperfusion injury, inflammatory bowel disease, autoimmune disease, viral infection, metabolic disease or cancer, or enhancing immune function, the method comprising administering an effective amount of a composition comprising the fusion protein as an active ingredient to a subject in need thereof.

Here, a detailed description of the present invention is provided.

The present invention provides a fusion protein comprising flagellin, fragment, or variant thereof, and a human IgG4 Fc variant, wherein the human IgG4 Fc variant has a mutation that prevents Fab arm exchange.

In the present invention, the flagellin can induce an immune response within the infected host when a flagellated bacterium is invaded. More specifically, Toll-like receptor 5 (TLR5), which is present on the surface of human cell membranes, interacts with flagellin to trigger intracellular signaling, leading to increased expression of the transcription factor NF-kB and activation of innate immune signaling as well as the regulation of acquired immune response.

The flagellin protein is well known, for example, from U.S. Pat. Nos. 6,585,980; 6,130,082; 5,888,810; 5,618,533; 4,886,748 and U.S. patent application publication US2003/0044429 A1, as well as from Donnelly et al. (2002) J. Biol. Chem. 43:4045. Most gram-negative bacteria express flagella, which are surface structures that provide motility. The flagellum is composed of a basal body, filament and hook that connects them. The filaments are made of long polymers of a single protein, flagellin, with a small cap protein at the end.

Flagellin polymerization is mediated by conserved domains at the N- and C-termini, while the hypervariable region in the middle of the flagellin protein has highly diverse sequences and lengths among different species.

In the present invention, the flagellin can be derived from any suitable bacteria. In the field, numerous flagellin genes have been cloned and sequenced, which can also be referred to. As an unrestricted source of the flagellin in the present invention, bacteria belonging to the genera Bacillus, Salmonella, Helicobacter, Vibrio, Serratia, Shigella, Treponema, Legionella, Borrelia, Clostridium, Agrobacterium, Bartonella, Proteus, Pseudomonas, Escherichia, Listeria, Yersinia, Campylobacter, Roseburia, or Marinobacter can be mentioned, and preferably, bacteria belonging to the genera Bacillus, Salmonella, or Vibrio are used.

More preferably, the flagellin in the present invention may be derived from Salmonella enteritidis, Salmonella typhimurium, Salmonella Dublin, Salmonella enterica, Helicobacter pylori, Vibrio cholera, Vibrio vulnificus, Vibrio fibrisolvens, Serratia marcesens, Shigella flexneri, Treponema pallidum, Borrelia burgdorferei, Clostridium difficile, Agrobacterium tumefaciens, Bartonella clarridgeiae, Proteus mirabilis, Bacillus subtilis, Bacillus cereus, Bacillus halodurans, Pseudomonas aeruginosa, Escherichia coli, Listeria monocytogenes, Yersinia pestis, Campylobacter spp, Roseburia spp, or Marinobacter spp, and
more preferably, from Salmonella enteritidis, Salmonella typhimurium, Salmonella Dublin, Salmonella enterica, Vibrio cholera, Vibrio vulnificus, Vibrio fibrisolvens, Bacillus subtilis, Bacillus cereus, or Bacillus halodurans,
and most preferably, from Bacillus subtilis.

The N-terminal and C-terminal constant regions of flagellin are well-known in the art. As understood by those skilled in the art, the size of the constant regions can vary somewhat depending on the source of the flagellin protein. Generally, the N-terminal constant domain contains approximately 170 or 180 N-terminal amino acids of the protein, while the C-terminal constant domain typically contains about 85 to 100 C-terminal amino acids. The central hypervariable region can vary considerably in size and sequence between bacteria, and most differences in molecular weight can be explained by this hypervariable region. Both the N- and C-terminal constant regions of flagellin proteins derived from various bacteria are known in the art, and flagellins from bacteria not yet characterized can easily be characterized using techniques known to those skilled in the art to determine the crystal structure of flagellin monomers.

In the present invention, the terms “flagellin,” “flagellin N-terminal constant region,” and “flagellin C-terminal constant region” include flagellin active fragments and variants derived from any of the bacteria exemplified herein. Furthermore, the wild-type flagellin or a portion thereof may be modified for increased safety and/or immunogenicity or as a result of cloning procedures or other laboratory manipulations, and such modifications (or variants) are also within the scope of the present invention.

In the present invention, the flagellin may include full-length flagellin or an active fragment. In addition, terms such as “flagellin”, “flagellin N-terminal constant region” and “flagellin C-terminal constant region” may include naturally occurring amino acid sequences. It may also include amino acid sequences that are substantially the same as or similar to amino acid sequences in naturally occurring flagellin, flagellin N-terminal constant regions, or flagellin C-terminal constant regions, respectively.

In the present invention, flagellin, flagellin N-terminal constant region, flagellin C-terminal constant region, or the “active fragment” of any other portion of flagellin may include at least about 50, 75, 100, 125, 150, 200, 250, or 300 adjacent amino acids and/or fewer than about 300, 250, 200, 150, 125, 100, or 75 adjacent amino acids. If the lower limit is smaller than the upper limit, a combination thereof may also be included. Such active fragment may represent a fragment capable of activating the TLR5 pathway in the host.

In certain embodiments, the active fragment can activate the TLR5 pathway as at least about 50%, 75%, 80%, 85%, 90%, or 95% of the full-length flagellin. The TLR5 pathway can be activated to the same or essentially the same extent as the full-length flagellin or flagellin site, or the TLR5 pathway can be activated to a higher degree compared to the full-length flagellin or flagellin site.

In the present invention, the active fragment may mean at least a portion of flagellin that exhibits activity in the TLR5 pathway. The term “at least a portion” may refer to a region of flagellin that exhibits activity in the TLR5 pathway in domains 0, 1, 2, and 3. Specifically, the active fragment may be flagellin that the hypervariable region has been removed. The hypervariable region may vary depending on the type of bacteria from which flagellin is derived, and among the entire sequence of specific flagellin, a sequence corresponding to the hypervariable region can be easily identified and removed by a person skilled in the art. For example, in the case of full-length flagellin including N-terminal domains 0, 1, 2, domain 3, and C-terminal domains 2, 1, 0, domain 3 or domains 2 and 3 may be the hypervariable region, and in the case of full-length flagellin including N-terminal domains 0, 1, domain 2, C-terminal domains 1, 0, domain 2 may be the hypervariable region. Alternatively, in the case of flagellin that does not contain a hypervariable region (e.g., flagellin derived from many Gram-positive bacteria may not contain a hypervariable region), a portion of the hinge region of the flagellin protein where folding occurs may have been partially removed.

The term “hypervariable region” used in the present invention may be expressed as a propeller domain or region, a hinge, a hypervariable region, a variable domain or region, and the like.

In present invention, the deletion of the hypervariable region may mean that the entire domain corresponding to the hypervariable region or the partial removal of some of the sequences within the hypervariable region.

In present invention, the active fragment may be flagellin in which the hypervariable region of wild-type flagellin is removed and an artificial sequence (i.e. a hinge or linker of the artificial sequence) is inserted into the removed hypervariable region.

In the present invention, the flagellin fragment of the present invention is a group consisting of a C-terminal domain 0, a C-terminal domain 1, a C-terminal domain 2, an N-terminal domain 2, an N-terminal domain 1, an N-terminal domain 0 of a wild type flagellin, and a region showing 80% or more amino acid sequence homology with each of the domains. And it may mean the fragment that exhibits TRL5 pathway activity while including one or more selected ones.

In certain embodiments, the active fragment can activate the TLR5 pathway as at least about 50%, 75%, 80%, 85%, 90%, or 95% of the full-length flagellin. The TLR5 pathway can be activated to the same or essentially the same extent as the full-length flagellin or flagellin site, or the TLR5 pathway can be activated to a higher degree compared to the full-length flagellin or flagellin site.

The present invention also includes proteins having the full-length sequence of wild-type flagellin as well as amino acid sequence variants thereof. In the present invention, a variant means a protein having a different sequence by deletion, insertion, non-conservative or conservative substitution, substitution of an amino acid analog or a combination of thereof. Amino acid substitutions that do not entirely alter the activity of the molecule (i.e., its ability to activate the TLR5 pathway) are known in the art.

In some cases, the variant of the present invention may be a full-length flagellin or fragment thereof modified by phosphorylation, sulfation, acrylation, glycosylation, methylation, farnesylation, or the like.

In certain embodiments, the flagellin or the variant of fragment thereof can activate the TLR5 pathway as at least about 50%, 75%, 80%, 85%, 90%, or 95% of the full-length flagellin or fragment thereof. The TLR5 pathway can be activated to the same or essentially the same extent by the full-length flagellin or flagellin site, or the TLR5 pathway can be activated to a higher degree compared to the full-length flagellin or flagellin site.

In the present invention, the flagellin, fragment, or variant thereof may be in the form of a fusion protein containing other polypeptides. For example, the flagellin may be a fusion protein containing one or more antigens. Non-limiting examples of such antigens may be included S. pneumoniae PspA1 antigen, S. pneumoniae PspA2 antigen, S. pneumoniae PspA3 antigen, S. pneumoniae PspA4 antigen, S. pneumoniae PspA5 antigen, and/or S. pneumoniae PspA6 antigen. Alternatively, for example, the flagellin may be in the form of a fusion protein in which one or more immunomodulatory substances are combined. The immunomodulatory substance may be included without limitation as long as it is known to increase the immune response in the art, and non-limiting examples thereof are interferon-α, interferon-β, interferon-γ, interferon-ω, interferon-τ, interleukin-1α, interleukin-1β, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-18, B-cell growth factor, CD40 ligand, TNF-α, TNF-β, CCL25, CCL28, or an active fragment thereof.

The term “percent (%) sequence identity” used in this invention is defined as the percentage of amino acid residues in a candidate sequence that are identical to amino acid residues in a reference polypeptide. After aligning the sequences and introducing gaps, any conservative substitutions are not considered part of the sequence identity, if necessary, to achieve the maximun percent sequence identity. Alignment for the purpose of determining percent amino acid homology can be performed using a variety of methods and methods within the skill of the art using, for example, publicly available computer software programs such as BLAST, BLAST-2, ALIGN) or Megalign (DNASTAR) software. One skilled in the art can determine appropriate parameters for measuring alignment, including any algorithms necessary to achieve maximal alignment over the entire length of the sequences being compared. For purposes herein, the percent (%) amino acid sequence identity of a given amino acid sequence A to a given amino acid sequence B or to a given amino acid sequence B is calculated as follows: 100 times fraction X/Y, where X is the number of amino acid residue scores identically matched by the sequence alignment program in program alignments of A and B, and Y is the total number of amino acid residues in B. It will be appreciated that if the length of amino acid sequence A is not equal to the length of amino acid sequence B, then the percent (%) amino acid sequence identity of A to B is not equal to the percent (%) amino acid sequence identity of B to A.

In a particular embodiment, the flagellin, fragment or variant thereof may be composed of an amino acid sequence selected from the group consisting of SEQ ID NO: 1 to 5, or an amino acid sequence showing at least 80% sequence homology with them.

In the present invention, the IgG4 Fc refers to a part of IgG4, excluding the variable regions of the heavy and light chains, the constant region 1 (CH1) of the heavy chain and constant region 1 (CL1) of the light chain, and including the constant regions 2 (CH2) and 3 (CH3) of the heavy chain, and optionally the hinge region. Moreover, in the present invention, the IgG4 Fc may be an extended IgG4 Fc that includes some or all of the constant region 1 (CH1) and/or constant region 1 (CL1) as well as the variable regions of the heavy and light chains, and which has substantially equivalent or improved properties compared to the wild-type immunoglobulin. It may also be an area where a considerably long part of the amino acid sequence corresponding to CH2 and/or CH3 has been removed.

On the other hand, IgG4 Fc may be selected to minimize the effector function by Fc when producing a fusion protein using a natural IgG Fc sequence. IgG4 is known to have relatively low effector function due to differences in amino acid sequences while showing a similar in vivo half-life to IgG1. However, despite the advantage of reduced effector function, it has been reported that the unique hinge sequence of IgG4 can cause Fab arm exchange between IgG4 molecules in vivo, leading to significant difficulties in using IgG4 as a fusion protein for therapeutic purposes (van der Neut Kolfschoten, et al., Science, 317:1554-1557. 2007). That is, when IgG4 Fc is used as a carrier for fusion protein, IgG4 existing in vivo and Fab arm exchange occur to form a hybrid with native IgG4 or to change the original structure by existing as a monomer to have low therapeutic activity. This is a common problem regardless of whether the IgG4 Fc fragment and physiologically active substance fusion are produced by genetic engineering methods or in vitro.

Therefore, the human IgG4 Fc variant included in the fusion protein of the present invention is characterized by including a mutation that can prevent Fab arm exchange.

A mutation that can prevent the arm exchange of Fab of IgG4 Fc described above can include a loss, insertion, or substitution of an amino acid that occurs in any one or more of the groups consisting of the hinge, CH2, and CH3 of the Fc.

Preferably, the mutation that can prevent the arm exchange of Fab of human IgG4 Fc is characterized by a mutation that confers inter-chain disulfide bond formation of human IgG4 Fc.

In the present invention, the “disulfide bond” referred to means a covalent bond formed between two sulfur atoms.

In one aspect of the present invention, the mutation that confers inter-chain disulfide bond formation of human IgG4 Fc is characterized by a mutation in the hinge. The hinge of human IgG4 Fc consists of a total of 12 amino acids (ESKYGPPCPSCP) (SEQ ID NO: 76), and the mutation in the hinge includes a loss, insertion, or substitution of an amino acid that occurs in the hinge sequence. Preferably, the mutation in the hinge includes a mutation in which the CPSC sequence among the 12 amino acids constituting the hinge of the human IgG4 Fc is substituted with CPPC and some amino acids are deleted or inserted, or another amino acid is substituted.

In a preferred aspect of the present invention, the human IgG4 Fc may be characterized by comprising a hinge consisting of 4 to 13 amino acids including a CPPC sequence. More specifically, the human IgG4 Fc may include a hinge consisting of a sequence of ESKYGPPCPPCP (SEQ ID NO: 77), SKYGPPCPPCP (SEQ ID NO: 78), KYGPPCPPCP (SEQ ID NO: 79), YGPPCPPCP (SEQ ID NO: 80), GPPCPPCP (SEQ ID NO: 81), PPCPPCP (SEQ ID NO: 82), PCPPCP (SEQ ID NO: 83), CPPCP (SEQ ID NO: 84), or CPPC (SEQ ID NO: 85).

In one aspect of the present invention, the human IgG4 Fc may be mutated in the hinge region to ensure formation of a disulfide bond between the two hinge regions. Preferably, the mutation in the human IgG4 Fc hinge region may include a mutation (S228P) of serine (Ser) to proline (Pro) at position 228 (according to EU numbering).

In another aspect of the present invention, the human IgG4 Fc can be characterized by the absence of certain amino acids in the hinge region of the wild-type IgG4 Fc, which prevents Fab arm exchange. Specific examples of amino acid deletions in the hinge region of IgG4 Fc that prevent Fab arm exchange are disclosed in KR20130063029 and the like.

In another aspect of the present invention, mutations that prevent Fab arm exchange in the IgG4 Fc may include S228P, R409K, or a combination thereof (according to EU numbering).

In another aspect of the present invention, the variant of the human IgG4 Fc may further comprise at least one amino acid mutation selected from the group consisting of Ser substituted with Pro at position 220 of the wild-type human IgG4 Fc (S220P), Gly substituted with Thr at position 223 (G223T), Pro substituted with His at position 224 (P224H), and Pro substituted with Thr at position 225 (P225T). Such amino acid mutations allow them to function as charge variants that affect protein stability without affecting the activity of wild-type human IgG4 Fc.

In another aspect of the present invention, a human IgG4 Fc variant capable of preventing a Fab arm exchange comprises an amino acid sequence selected from the group consisting of SEQ ID NO 6 to 11 or an amino acid sequence having a sequence homology of 80% or more.

Furthermore, in the present invention, the human IgG4 Fc variant is a mutation that prevents Fab arm exchange, and also includes a mutein of the IgG4 Fc variant. In the present invention, a derivative of an IgG4 Fc variant means having a different sequence due to deletion, insertion, non-conservative or conservative substitution of one or more amino acid residues in the amino acid sequence, or a combination thereof. In addition, various types of derivatives are possible, such as removing some amino acids at the N-terminus of native Fc or adding a methionine residue to the N-terminus of native Fc. In addition, in order to eliminate the effector function, a complement binding site, eg, a C1q binding site, may be removed, or an ADCC site may be removed. Techniques for preparing such Fc region sequence derivatives are disclosed in International Patent Publication No. 97/34631, International Patent Publication No. 96/32478, and the like.

Amino acid substitutions in proteins and peptides that do not globally alter the activity of the molecule are well known in the art. The most commonly occurring substitutions are those between amino acid residues Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly.

In some cases, the human IgG4 Fc variant described herein can be modified by phosphorylation, sulfation, acrylation, glycosylation, methylation, farnesylation, acetylation, amidation, or other modifications.

The induced variants of the human IgG4 Fc variant described above exhibit the same biological activity as the human IgG4 Fc variant, but have increased structural stability under conditions such as temperature and pH.

In one embodiment of the present invention, a fusion protein of a derivative in which the C-terminal 3^{rd} amino acid of wild-type human IgG4 Fc is substituted from Leu to Pro (including an amino acid mutation that prevents Fab arm exchange in the hinge region) and flagellin is prepared. As a result of evaluating the effect, it was confirmed that there was no difference between the flagellin fusion protein containing the C-terminal sequence of wild-type human IgG4 Fc and the TLR5 agonist activity.

That is, in the present invention, the derivative of the human IgG4 Fc mutant may additionally include a mutation that does not affect the activity of the fusion protein and improves the stability of the protein. More specifically, the derivative of the wild-type human IgG4 Fc may be a derivative in which the C-terminal 3rd amino acid is substituted with Pro in Leu.

Furthermore, the human IgG4 Fc variant may exist in the form of natural glycosylation, increased or decreased glycosylation compared to natural forms, or in a form with removed glycosylation. Common methods such as chemical, enzymatic, and genetic engineering using microorganisms can be used to increase, decrease, or remove glycosylation in these Fc regions. The deglycosylated human IgG4 Fc variant has significantly reduced complement (C1q) binding affinity and reduced or eliminated antibody-dependent or complement-dependent cell cytotoxicity, thereby not inducing unnecessary immune responses in the body. In this regard, Fc variants of human IgG4 with removed or deglycosylated forms are more suitable for their original purpose as drug carriers.

In the present invention, “deglycosylation” refers to a human IgG4 Fc variant where the sugar has been removed by an enzyme, while “aglycosylation” refers to a variant that is not glycosylated, produced in prokaryotes, preferably E. coli.

In the present invention, the fusion protein may be one in which the N-terminus or C-terminus of flagellin, fragment, or variant thereof is linked to the N-terminus or C-terminus of the human IgG4 Fc variant. Specifically, the N-terminus of flagellin, fragment, or variant thereof is linked to the C-terminus of a human IgG4 Fc variant, or the C-terminus of the flagellin, fragment, or variant thereof is a human IgG4 Fc variant. It may be bonded to the N-terminus. Preferably, the C-terminus of flagellin, fragment, or variant thereof may be linked to the N-terminus of a human IgG4 Fc variant.

Meanwhile, each component constituting the fusion protein in the present invention, namely flagellin, fragment or variant thereof, and human IgG4 Fc variant may be directly connected or connected through a linker. Generally, the term “linker” refers to a nucleic acid, amino acid, or non-peptide residue that can be inserted between one or more molecules. For example, linkers can be used to facilitate manipulation by providing desired regions of interest between components. Linkers can also be provided to enhance expression of the fusion protein from transformants and to reduce steric hindrance so that the component can assume its optimal tertiary structure and/or interact properly with the target molecule The linker sequence may include one or more amino acids naturally linked to the receptor component. It maybe also additional sequence to enhance expression of the fusion protein, or to provide a desired site of particular interest, and/or to enhance the interaction of the component with its target molecule.

Ideally, the mentioned linker can increase the flexibility of the fusion protein without interfering with the structure of each component of the fusion protein. In some embodiments, the linker residue is a peptide linker with 2 to 100 amino acid residues in length. Exemplary linkers include linear peptides with at least 2 amino acid residues such as Gly-Gly (SEQ ID NO: 64), Gly-Ala-Gly (SEQ ID NO: 65), Gly-Pro-Ala (SEQ ID NO: 66), Gly(G)n (SEQ ID NO: 67), and Gly-Ser (GS) (SEQ ID NO: 68) linkers. The GS linker disclosed in this specification includes (GS) n (SEQ ID NO: 68), (GSGSG)n (SEQ ID NO: 69), (G2S)n (SEQ ID NO: 70), G2S2G (SEQ ID NO: 71), (G2SG) n (SEQ ID NO: 72), (G3S) n (SEQ ID NO: 73), (G4S) n (SEQ ID NO: 12), (GGSGG)nGn (SEQ ID NO: 74), GSG4SG4SG (SEQ ID NO: 75), and (GGGGS)n (SEQ ID NO: 12), but is not limited thereto, wherein n is an integer of 1 or more. An example of a (G)n linker is the G9 linker, and an example of a (GGGGS)n (SEQ ID NO: 12) linker includes GGGGS (SEQ ID NO: 12) or (GGGGS)3 (SEQ ID NO: 13) linker composed of suitable linear peptides including polyglycine, polyserine, polyproline, polyalanine, and oligopeptides composed of alanine and/or serine and/or proline and/or glycine amino acid residues. The linker residue can be used to connect the constituent components of the fusion protein disclosed herein.

In the present invention, the linker may be composed of an amino acid sequence with SEQ ID NO: 12 or SEQ ID NO: 13.

The fusion protein disclosed in this invention may or may not include a signal peptide that functions to secrete the fusion protein from the host cell. A nucleic acid sequence encoding a signal peptide can be operably linked to a nucleic acid sequence encoding a protein of interest. In some embodiments, the fusion protein includes a signal peptide. In some embodiments, the fusion protein does not include a signal peptide.

Furthermore, the fusion protein described in this invention may include modified forms of protein-binding peptides. For example, the fusion protein component can undergo post-translational modifications such as glycosylation, sialylation, acetylation, and phosphorylation on any protein-binding peptide.

Unless otherwise stated, the fusion protein of this invention is administered as a polypeptide (or a nucleic acid encoding the polypeptide), which is not a part of a live, attenuated, or recombinant bacterial or viral vector vaccine. Additionally, unless otherwise specified, the fusion protein of this invention is a purified fusion protein and for example, it is not incorporated into the flagellum.

In the present invention, “fusion” refers to the fusion of two molecules with different or identical functions or structures, by any physical, chemical, or biological means that allow peptide binding. The fusion protein or polypeptide constituting the fusion protein can be produced by chemical peptide synthesis methods disclosed in the art, or by cloning the gene encoding the fusion protein by PCR (polymerase chain reaction) amplification or synthesis using the disclosed methods and expressing it in an expression vector.

In a specific embodiment of the present invention, the fusion protein may include an amino acid sequence of SEQ ID NO: 49, SEQ ID NO: 51, SEQ ID NO: 53, SEQ ID NO: 55, SEQ ID NO: 57, SEQ ID NO: 59, SEQ ID NO: 61, or SEQ ID NO: 63.

The present invention also provides a polynucleotide comprising a nucleotide sequence encoding the fusion protein.

The above polynucleotide is not specifically limited to any particular combination of nucleotides that can encode the polypeptide of the present invention. The polynucleotide may be provided as a single-stranded or double-stranded nucleic acid molecule, including DNA, cDNA, and RNA sequences.

Preferably, the polynucleotide may have a nucleotide sequence of SEQ ID NO: 48, SEQ ID NO: 50, SEQ ID NO: 52, SEQ ID NO: 54, SEQ ID NO: 56, SEQ ID NO: 58, SEQ ID NO: 60, or SEQ ID NO: 62.

The present invention also provides a vector comprising the polynucleotide.

The vectors of the present invention include, but are not limited to, plasmid vectors, cosmid vectors, bacterial phage vectors, and viral vectors. The vector of the present invention may be a conventional cloning vector or expression vector. The expression vectors can be manufactured in various ways depending on the purpose, including signal sequences or leader sequences for membrane targeting or secretion in addition to expression control sequences such as promoters, operators, initiation codons, termination codons, polyadenylation signals, and enhancers. The polynucleotide sequences according to the present invention may be operably linked to an expression control sequence, and the operably linked gene sequence and expression control sequence can be included in one expression vector that contains a selectable marker and a replication origin. To be “operably linked” is to be connected in such a way as to enable gene expression when appropriate molecules are linked to expression regulatory sequences, wherein one nucleic acid fragment is linked to another nucleic acid fragment to alter its function or expression.

“Expression control sequence” refers to a DNA sequence that controls the expression of a polynucleotide sequence operably linked to it in a particular host cell. Such regulatory sequences include promoters to effect transcription, optional operator sequences to regulate transcription, sequences encoding suitable mRNA ribosome binding sites, and sequences to control termination of transcription and translation. In addition, the vector includes a selectable marker for selecting a host cell containing the vector, and an origin of replication in the case of a replicable vector.

The present invention also provides a transfectant transformed with the vector.

Transformation with the vector may be performed by a transfectant technique known to those skilled in the art. Preferably, microprojectile bombardment, electroporation, calcium phosphate (CaPO₄) precipitation, calcium chloride (CaCl₂)) precipitation, PEG-mediated fusion, microinjection and a liposome-mediated method may be used.

The term ‘transfectant’ may be used interchangeably with ‘host cell’ and the like, and is introduced into cells by any methods (e.g., electroporation, calcium phosphatase precipitation, microinjection, transformation, viral infection, etc.) means a prokaryotic or eukaryotic cell containing heterologous DNA.

In the present invention, the transfectant include single-cell organisms of all types commonly used in the cloning field, including prokaryotic microorganisms such as various bacteria (e.g., Clostridium spp., E. coli, etc.), lower eukaryotic microorganisms such as yeasts, and higher eukaryotic cells derived from plants, insects, mammals, etc., and are not limited thereto. Depending on the host cell, the expression level and modification of the protein appear differently, so a person skilled in the art can select and use the most suitable host cell for the purpose.

The present invention also provides a pharmaceutical composition comprising the fusion protein as an active ingredient

According to one embodiment of the present invention, it was confirmed that the fusion protein exhibits significantly improved TLR5 pathway activating ability compared to wild-type flagellin. In addition, it was confirmed that the TLR5 pathway activating ability was significantly improved compared to the fusion protein in which wild-type IgG4 Fc and flagellin were fused. Accordingly, the fusion protein of the present invention may exhibit preventive, improvable, or therapeutic effects on diseases, syndromes, etc. known to be preventable, improvable, or treated through activation of the TLR5 pathway.

Diseases or syndromes known to be preventable, improvable or curable through activation of the TLR5 pathway may be radiation-induced damage, reperfusion injury, inflammatory bowel disease, autoimmune disease, viral infection, aging, reduced immune function, or cancer.

Therefore, the pharmaceutical composition comprising the fusion protein of the present invention can be characterized as a preventative or therapeutic agent for radiation-induced damage, reperfusion injury, inflammatory bowel disease, autoimmune diseases, viral infections, aging, immune dysfunction, or cancer.

In particular, as the fusion protein of the present invention is expected to exhibit preventative, therapeutic, or improving effects for diseases that will be discovered in the future through TLR5 pathway activation, the scope of diseases targeted by the pharmaceutical composition of the present invention is not particularly limited.

The relevance of TLR5 pathway activation to the treatment of radiation-induced damage can be found in KR20067010934A and the relevance of TLR5 pathway activation to the treatment of tissue damage caused by reperfusion can be found in U.S. Pat. No. 8,324,163. The relevance of TLR5 pathway activation to the treatment of inflammatory bowel disease can be found in U.S. Pat. No. 7,361,733, and the relevance of TLR5 pathway activation to the treatment of autoimmune diseases can be found in EP03010523B1. The relevance of TLR5 pathway activation to the treatment of viral infections can be found in U.S. Pat. No. 9,872,895, and the relevance of TLR5 pathway activation to the diseases caused by aging can be found in KR20150049811A. The relevance of TLR5 pathway activation to immune enhancement can be found in WO17031280A1, and the relevance of TLR5 pathway activation to cancer treatment can be found in KR20177005615A.

In the present invention, radiation-induced damage may be gastrointestinal syndrome or hematopoietic syndrome caused by radiation exposure.

The diseases caused by aging referred to in this invention may include alopecia, cataracts, hernias, ulcerative colitis, osteoporosis, or osteomalacia.

In this invention, the cancer referred to may include breast cancer, lung cancer, colon cancer, kidney cancer, liver cancer, ovarian cancer, prostate cancer, testicular cancer, urological cancer, lymphatic system cancer, rectal cancer, pancreatic cancer, esophageal cancer, stomach cancer, cervical cancer, thyroid cancer, skin cancer, leukemia, acute lymphocytic leukemia, acute myelogenous leukemia, B-cell lymphoma, T-cell lymphoma, Hodgkin's lymphoma, non-Hodgkin's lymphoma, mycosis fungoides, tissue lymphoma, and Burkitt's lymphoma, acute and chronic myelogenous leukemia, myelodysplastic syndrome, myeloid leukemia, preleukemia, neuroblastoma, ganglioma, neurofibroma, fibrosarcoma, leiomyosarcoma, osteosarcoma, melanoma, squamous cell carcinoma, basal cell carcinoma, thyroid follicular carcinoma, teratoma, or gastrointestinal cancer.

The pharmaceutical composition of the present invention can be formulated in various dosage forms depending on the administration route, using a pharmaceutically acceptable carrier in accordance with a method disclosed in the art, in addition to the fusion protein. The term “pharmaceutically acceptable” refers to a non-toxic substance that is physiologically acceptable and does not interfere with the action of the active ingredient, and does not usually cause allergic reactions or similar reactions such as gastrointestinal disturbances or dizziness when administered to humans. Examples of such carriers include all types of solvents, dispersants, aqueous or oil-in-water emulsions, aqueous compositions, liposomes, microbeads, and microsomes.

The administration route can be either oral or non-oral. Non-oral routes of administration may include but are not limited to intravenous, intramuscular, intraarterial, intramedullary, intrathecal, intracardiac, transdermal, subcutaneous, intraperitoneal, intranasal, intestinal, topical, rectal or intrarectal.

When the pharmaceutical composition of the present invention is administered orally, it can be formulated in various forms such as powders, granules, tablets, capsules, liquids, gels, syrups, suspensions, wafers, and the like according to methods known in the art using suitable oral delivery vehicles. Examples of suitable vehicles include sugars such as lactose, dextrose, sucrose, sorbitol, mannitol, xylitol, erythritol, and maltitol, as well as starches such as corn starch, wheat starch, rice starch, and potato starch, and cellulose derivatives such as cellulose, methylcellulose, sodium carboxymethylcellulose, and hydroxypropyl methylcellulose, and fillers such as gelatin and polyvinylpyrrolidone. In some cases, cross-linked polyvinylpyrrolidone, carrageenan, alginic acid, or sodium alginate may be added as a disintegrant. Additionally, the pharmaceutical composition may further include anti-adherents, lubricants, wetting agents, fragrances, emulsifiers, and preservatives as needed.

In addition, when administered non-orally, the pharmaceutical composition of the present invention can be formulated in the form of injectables, transdermal delivery agents, and nasal inhalants, along with suitable non-oral vehicles, according to methods known in the art. For injectables, the composition must be sterilized and protected from contamination by microorganisms such as bacteria and fungi. Suitable vehicles for injectables may include, but are not limited to, water, ethanol, polyols such as glycerol, propylene glycol, and liquid polyethylene glycol, mixtures thereof, and/or solvents or dispersing agents containing plant oils. Preferably, suitable vehicles include Hank's solution, Ringer's solution, PBS (phosphate-buffered saline) containing triethanolamine, or sterile injectable water, 10% ethanol, appearance solutions such as 40% propylene glycol and 5% dextrose. To protect the injectables from microbial contamination, various antimicrobial and antifungal agents such as parabens, chlorobutanol, phenol, sorbic acid, and thimerosal can be added. Additionally, most injectables can include isotonic agents such as dextrose or sodium chloride.

For the case of transdermal administration, the form of the pharmaceutical composition can include ointments, creams, lotions, gels, topical solutions, pastes, liniments, aerosols, and the like. The term “transdermal administration” as used herein refers to the delivery of the pharmaceutical composition to the skin locally, so that an effective amount of the active ingredient contained in the pharmaceutical composition is delivered into the skin. For example, the pharmaceutical composition of the present invention can be administered by preparing it in injectable form and injecting it lightly into the skin using a fine 30 gauge needle (prick), or by direct application to the skin. These forms are described in prescription references commonly known in pharmaceutical chemistry.

For the case of inhalation administration, the compound used according to the present invention can be conveniently delivered in aerosol spray form from a pressurized pack or a nebulizer with a suitable propellant, such as dichlorodifluoromethane, trichlorofluoromethane, dichlorotetrafluoroethane, carbon dioxide, or other suitable gas. The dosage units can be determined by providing a valve to deliver a metered amount. For example, gelatin capsules and cartridges for use in an inhaler or insufflator can be formulated to contain a powder mix of the compound and a suitable powder base, such as lactose or starch.

As other pharmaceutically acceptable carriers, reference may be made to those known in the art.

In addition, the pharmaceutical composition according to the present invention may further include one or more of a buffer (for example, saline or PBS), a carbohydrate (for example, glucose, mannose, sucrose, or dextran), an antioxidant, a bacteriostat, a chelating agent (for example, EDTA or glutathione), an adjuvant (for example, aluminum hydroxide), suspending agents, thickening agents and/or preservatives.

Furthermore, the pharmaceutical composition of the present invention can be formulated using methods known in the art to provide rapid, sustained or delayed release of the active ingredient after administration to a mammal.

In addition, the pharmaceutical composition of the present invention can be administered in combination with a known substance having the effect of preventing or treating each of the diseases listed above.

The present invention also provides a vaccine adjuvant comprising the fusion protein as an active ingredient.

One of the most important requirements for a vaccine adjuvant is to have immunomodulatory functions such as regulating the expression of co-stimulatory molecules on antigen-presenting cells and cytokine secretion by antigen-specific T cells.

However, PRRs such as TLR5 are distributed on the cell surface or cytoplasm of host cells, induce ‘innate immune response’ by stimulation of various PAMPs, regulating subsequent ‘adaptive immune responses.’ Therefore, TLR5 agonists can serve as targets for various ‘immunomodulators,’ especially ‘vaccine adjuvants.’

Accordingly, the fusion protein of the present invention capable of activating the TLR5 pathway activates the TLR5 pathway to enhance innate and acquired immune responses, thereby significantly improving the host's immune ability against co-administered antigens.

The vaccine adjuvant of the present invention can be prepared by a conventional method well known in the art, and may optionally further include various additives that can be used in vaccine preparation in the art.

The present invention also provides a use of the fusion protein for the preparation of an agent for treating radiation exposure-induced damage, reperfusion injury, inflammatory bowel disease, autoimmune disease, viral infection, metabolic disease or cancer, or enhancing immune function.

The present invention provides a method for treating radiation exposure-induced damage, reperfusion injury, inflammatory bowel disease, autoimmune disease, viral infection, metabolic disease or cancer, or enhancing immune function, the method comprising administering an effective amount of a composition comprising the fusion protein as an active ingredient to a subject in need thereof.

The term ‘effective amount’ in the present invention refers to an amount that exhibits an effect of improving, treating, detecting, diagnosing, or inhibiting or reducing the progression of radiation-induced damage, reperfusion injury, inflammatory bowel disease, autoimmune disease, viral infection, metabolic disease, aging, enhancing immune function, or cancer when administered to a subject, and the ‘subject’ refers to an animal, It may be preferably a mammal, especially an animal including a human, and may also be a cell, tissue, organ, etc. derived from an animal. The subject may be a patient in need of the effect.

The term ‘treatment’ in the present invention broadly refers to improving symptoms radiation-induced damage, reperfusion injury, inflammatory bowel disease, autoimmune disease, viral infection, metabolic disease, aging, enhancing immune function, or cancer and may include curing or substantially preventing the disease or improving the condition caused by the disease. The treatment may encompass relieving one or most of the symptoms caused by the disease or preventing or curing them, but is not limited thereto.

The term “comprising” used in this specification means the same as “including” or “characterized by”, and does not exclude additional components or method steps that are not specifically mentioned in the composition or method according to the present invention. Additionally, the term “consisting of” means that any additional elements, steps or components that are not specifically mentioned are excluded. The term “essentially consisting of” means that the composition or method may include additional components or steps that do not substantially affect the basic characteristics of the composition or method, in addition to the substances or steps that are specifically mentioned.

### Effect of the Invention

The fusion protein provided by the present invention not only exhibits significantly superior Toll-like receptor 5 (TLR5) pathway activation compared to wild-type flagellin, fragment, or variant thereof, but also shows significantly better TLR5 pathway activation compared to a protein fused with wild-type IgG4 Fc and flagellin. Therefore, the fusion protein can be extremely useful for developing therapeutic agents and/or vaccine adjuvants for diseases that can be prevented, improved, or treated through TLR5 pathway activation.

### BRIEF DESCRIPTION OF THE FIGURES

FIG. 1 shows the results of evaluating the TLR5 activation ability of fusion protein 1 and 4 produced in an embodiment of the present invention.

FIG. 2 shows the results of evaluating the TLR5 activation ability of fusion protein 1 and 2 produced in an embodiment of the present invention.

FIG. 3 shows the results of evaluating the TLR5 activation ability of fusion protein 4 to 7 produced in an embodiment of the present invention.

FIG. 4 shows the results of evaluating the TLR5 activation ability of fusion protein 6 to 11 produced in an embodiment of the present invention.

FIG. 5 shows the results of evaluating the TLR5 activation ability of fusion protein 6 produced in an embodiment of the present invention.

FIG. 6 shows the results of the protein-protein interaction between the fusion protein 6 and zTLR5, analyzed by biolayer interferometry, and the complex formation between the fusion protein and TLR5 analyzed by gel-filtration chromatography in the embodiments of the present invention.

FIG. 7 shows the evaluation of the TLR5 activation ability of the fusion protein 6 at various concentrations and the determination of its EC50 value in the embodiments of the present invention.

FIG. 8 shows the evaluation of the TLR5 activation abilities of the fusion protein 6 and 12 at various concentrations in the embodiments of the present invention.

FIG. 9 shows the analysis of drug absorption according to the time elapsed after intranasal administration of the SEQ ID NO: 63 fusion protein and wild-type Bacillus subtilis flagellin (BsFlagellin) using fluorescence imaging (A: SEQ ID NO: 63 fusion protein, B: wild-type Bacillus subtilis flagellin).

### DETAILED DESCRIPTION FOR CARRYING OUT THE INVENTION

The following detailed description of the embodiments of the present invention is provided. However, the following embodiments are for illustrative purposes only, and the present invention is not limited by them.

### 1. Experimental Method

In the present invention, various forms of fusion proteins were produced to compare the effect of Fab arm exchange prevention in human IgG4 Fc mutant-flagellin fusion proteins with other forms of Fc-flagellin fusion proteins. The flagellin used in the production of fusion proteins was Bacillus subtilis flagellin (BsFlagellin).

### (1) DNA Cloning

Twelve types of plasmids were created:
1. pFUSE-hIgG4-fc2-bsFlagellin-partial hinge (MSP303)
2. pFUSE-hIgG4-fc2-bsFlagellin-partial hinge (MSP304)
3. pFUSE-hIgG4-fc2-bsFlagellin-partial hinge-NL
4. pFUSE-hIgG4-fc2-bsFlagellin-full hinge-CPSC (MSP305)
5. pFUSE-hIgG4-fc2-bsFlagellin-full hinge-CPSC-NL
6. pFUSE-hIgG4-fc2-bsFlagellin-full hinge-CPPC (MSP306)
7. pFUSE-hIgG4-fc2-bsFlagellin-full hinge-CPPC-NL
8. pFUSE-hIgG4-fc2-bsFlagellin-full hinge-CPPC-S220P
9. pFUSE-hIgG4-fc2-bsFlagellin-full hinge-CPPC-G223T
10. pFUSE-hIgG4-fc2-bsFlagellin-full hinge-CPPC-P224H
11. pFUSE-hIgG4-fc2-bsFlagellin-full hinge-CPPC-P225T
12. pFUSE-hIgG4-Fc2-bsFlagellin-full hinge-CPPC-LGK

The composition of the human IgG4 Fc-flagellin fusion protein variants encoded by the plasmids 1 to 12 described above are shown in Table 1:

TABLE 1

| | Composition | | | | |
|---|---|---|---|---|---|
| No | Flagellin | Linker | Hinge | Fc | Other |
| 1 | BsFlagellin | GGGGS | PPCPSCP | hIgG4 | — |
| | | (SEQ ID NO: 12) | (SEQ ID NO: 86) | | |
| 2 | | (GGGGS) X 3 | PPCPSCP | | — |
| | | (SEQ ID NO: 13) | (SEQ ID NO: 86) | | |
| 3 | | N/A | PPCPSCP | | — |
| | | | (SEQ ID NO: 86) | | |
| 4 | | GGGGS | ESKYGPPCPSCP | | — |
| | | (SEQ ID NO: 12) | (SEQ ID NO: 76) | | |
| 5 | | N/A | ESKYGPPCPSCP | | — |
| | | | (SEQ ID NO: 76) | | |
| 6 | | GGGGS | ESKYGPPCPPCP | | The C-terminal 3^{rd} |
| | | (SEQ ID NO: 12) | (S228P) | | sequence of hIgG4 Fc |
| | | | (SEQ ID NO: 77) | | has a point mutation |
| | | | | | to P |
| 7 | | N/A | ESKYGPPCPPCP | | — |
| | | | (S228P) | | |
| | | | (SEQ ID NO: 77) | | |
| 8 | | GGGGS | EPKYGPPCPPCP | | Charge variants |
| | | (SEQ ID NO: 12) | (S220P, S228P) | | |
| | | | (SEQ ID NO: 87) | | |
| 9 | | GGGGS | ESKYTPPCPPCP | | |
| | | (SEQ ID NO: 12) | (G223T, S228P) | | |
| | | | (SEQ ID NO: 88) | | |
| 10 | | GGGGS | ESKYGHPCPPCP | | |
| | | (SEQ ID NO: 12) | (P224H, S228P) | | |
| | | | (SEQ ID NO: 89) | | |
| 11 | | GGGGS | ESKYGPTCPPCP | | |
| | | (SEQ ID NO: 12) | (P225T, S228P) | | |
| | | | (SEQ ID NO: 90) | | |
| 12 | | GGGGS | ESKYGPPCPPCP | | — |
| | | (SEQ ID NO: 12) | (S228P) | | |
| | | | (SEQ ID NO: 77) | | |

The hinge region of human IgG4 Fc is composed of a total of 12 amino acids (ESKYGPPCPSCP) (SEQ ID NO: 76). The human IgG4 Fc included in the fusion proteins of the above 1 to 3 contains a truncated form (PPCPSCP) (SEQ ID NO: 86), where some of the amino acids that make up the hinge region of human IgG4 Fc are missing, which is included in the human IgG4 Fc sequence included in the common plasmids. The sequences of the full-length wild-type hinge, which include all 12 amino acids, or full-length hinge sequences that include one or more amino acid mutations are included in the above 5 to 12.

The primers used for the DNA production in the above 1 to 12 are listed in Table 2 below.

TABLE 2

| No | Used Primer | |
|---|---|---|
| 1. pFUSE-hIgG4-fc2- | Forward | 5′-C CGG ATA TCG ATG AGA ATT AAC CAC AAT ATT |
| bsFlagellin-partial hinge | | GCA GCA CTT AAC-3′ |
| (MSP303) | | (SEQ ID NO: 14) |
| | Reverse | 5′-GAC CAT GGC AGA CCC TCC GCC ACC ACG TAA |
| | | TAA TTG AAG TAC GTT TTG AGG CTG-3′ |
| | | (SEQ ID NO: 15) |
| 2. pFUSE-hIgG4-fc2- | Forward | 5′-C CGG ATA TCG ATG AGA ATT AAC CAC AAT ATT |
| bsFlagellin-partial hinge | | GCA GCA CTT AAC-3′ |
| (MSP304) | | (SEQ ID NO: 16) |
| | Reverse | 5′-GAC CAT GGC AGA CCC TCC GCC ACC AGA CCC |
| | | TCC GCC ACC AGA CCC TCC GCC ACC ACG TAA TAA |
| | | TTG AAG TAC GTT TTG AGG CTG-3′ |
| | | (SEQ ID NO: 17) |
| 3. pFUSE-hIgG4-fc2- | Forward | 5′-C CGG ATA TCG ATG AGA ATT AAC CAC AAT ATT |
| bsFlagellin-partial hinge-NL | | GCA GCA CTT AAC-3′ |
| | | (SEQ ID NO: 18) |
| | Reverse | 5′-TCA GAT CTA ACC ATG GCA CGT AAT AAT TGA |
| | | AG-3′ |
| | | (SEQ ID NO: 19) |
| 4. pFUSE-hIgG4-fc2- | Forward | 5′-TAT ATC CAT GGT TAG ATC TGA ATC CAA ATA |
| bsFlagellin-full hinge-CPSC | | CGG TCC CCC ATG CCC ATC-3′ |
| (MSP305) | | (SEQ ID NO: 20) |
| | Reverse | 5′-TAT ATG CTA GCA CTC ATT TAC CCA GAG ACA |
| | | GGG AGA G-3′ |
| | | (SEQ ID NO: 21) |
| 5. pFUSE-hIgG4-fc2- | Forward | 5′-C CGG ATA TCG ATG AGA ATT AAC CAC AAT ATT |
| bsFlagellin-full hinge-CPSC-NL | | GCA GCA CTT AAC-3′ |
| | | (SEQ ID NO: 22) |
| | Reverse | 5′-TCA GAT CTA ACC ATG GCA CGT AAT AAT TGA |
| | | AG-3′ |
| | | (SEQ ID NO: 23) |
| 6. pFUSE-hIgG4-fc2- | Forward | 5′-TAT ATC CAT GGT TAG ATC TGA ATC CAA ATA |
| bsFlagellin-full hinge-CPPC | | CGG TCC CCC ATG CCC ACC TTG CCC AGC ACC |
| (MSP306) | | TGA-3′ |
| | | (SEQ ID NO: 24) |
| | Reverse | 5′-TAT ATG CTA GCA CTC ATT TAC CCA GAG ACA |
| | | GGG AGA G-3′ |
| | | (SEQ ID NO: 25) |
| 7. pFUSE-hIgG4-fc2- | Forward | 5′-C CGG ATA TCG ATG AGA ATT AAC CAC AAT ATT |
| bsFlagellin-full hinge-CPPC-NL | | GCA GCA CTT AAC-3′ |
| | | (SEQ ID NO: 26) |
| | Reverse | 5′-TCA GAT CTA ACC ATG GCA CGT AAT AAT TGA |
| | | AG-3′ |
| | | (SEQ ID NO: 27) |
| 8. pFUSE-hIgG4-fc2- | Forward | 5′-GGA GGG TCT GCC ATG GTT AGA TCT GAA CCC |
| bsFlagellin-full hinge-CPPC- | | AAA TAC GGT CCC CCA TGC CCA CCT TGC-3′ |
| S220P | | (SEQ ID NO: 28) |
| | Reverse | 5′-TAT ATG CTA GCA CTC ATT TAC CCA GAG ACA |
| | | GGG AGA G-3′ |
| | | (SEQ ID NO: 29) |
| 9. pFUSE-hIgG4-fc2- | Forward | 5′-GGA GGG TCT GCC ATG GTT AGA TCT GAA TCC |
| bsFlagellin-full hinge-CPPC- | | AAA TAC ACT CCC CCA TGC CCA CCT TGC-3′ |
| G223T | | (SEQ ID NO: 30) |
| | Reverse | 5′-TAT ATG CTA GCA CTC ATT TAC CCA GAG ACA |
| | | GGG AGA G-3′ (SEQ ID NO: 31) |
| 10. pFUSE-hIgG4-fc2- | Forward | 5′-GGA GGG TCT GCC ATG GTT AGA TCT GAA TCC |
| bsFlagellin-full hinge-CPPC- | | AAA TAC GGT CAC CCA TGC CCA CCT TGC-3′ |
| P224H | | (SEQ ID NO: 32) |
| | Reverse | TAT ATG CTA GCA CTC ATT TAC CCA GAG ACA GGG |
| | | AGA G-3′ |
| | | (SEQ ID NO: 33) |
| 11. pFUSE-hIgG4-fc2- | Forward | 5′-GGA GGG TCT GCC ATG GTT AGA TCT GAA TCC |
| bsFlagellin-full hinge-CPPC- | | AAA TAC GGT CCC ACA TGC CCA CCT TGC-3′ |
| P225T | | (SEQ ID NO: 34) |
| | Reverse | 5′-TAT ATG CTA GCA CTC ATT TAC CCA GAG ACA |
| | | GGG AGA G-3′ |
| | | (SEQ ID NO: 35) |
| 12. pFUSE-hIgG4-fc2- | Forward | 5′-GAC AAG GAT ATC GAT GAG AAT TAA CCA CAA |
| bsFlagellin-full hinge-CPPC | | TAT TGC AGC ACT TAA CAC-3′ |
| (MSP306L) | | (SEQ ID NO: 36) |
| | Reverse | 5′-GAC GCT AGC TCA TTT ACC CAG AGA CAG GGA |
| | | GAG GC-3′ |
| | | (SEQ ID NO: 37) |

The cloning was performed according to the following steps:

### [PCR]

1) Using the primers shown in Table 2, PCR was performed with pFUSE-hIgG4-Fc2-BS1 as the template DNA to produce CPSC and CPPC PCR products.
2) 10×DNA dye was added to the PCR fragment DNA and gel electrophoresis was performed to confirm the size and presence of bands in the PCR product. Gel extraction was then performed using MEGAquick-spin Plus (17290).

### [Enzyme Restriction]

3) The PCR fragment and Template DNA from step 2) were treated with restriction enzymes. After enzyme cutting was completed for each plasmid, 10×DNA dye was added, and gel electrophoresis was performed to confirm the size and presence of bands in the PCR fragment and Template DNA products. Gel extraction was then performed using MEGAquick-spin Plus (17290).

### [Ligation]

4) Ligation was performed at a molar ratio of Vector (Template DNA):Insert (PCR fragment)=1:5. The ligation process was carried out according to the manual of the Takara T4 DNA Ligase Kit (2011A).

### [Transformation]

5) After thawing DH5a competent cells on ice for 1 minute, leave the Zeocin(+) TB plate in a 37° C. incubator.
6) Add the Ligation Product to 10% volume of the thawed DH5a competent cells and adjust the total amount.
7) Give a heat shock for 30 seconds in a 42° C. water bath.
8) Leave on ice for 5 minutes after heat shock.
9) Spread the transformed to E. coli on Zeocin (+) TB Plate.
10) Grow colonies overnight and pick a colony.
11) Perform Mini Prep after 18 hours of 3 mL Inoculation of the picked colony in Zeocin (+) LB media.
12) DNA sequencing is performed to confirm the nucleotide sequence of the cloned plasmids.

The DNA nucleotide sequence and protein amino acid sequence of the fusion protein from 1 to 12 are shown in Table 3.

TABLE 3

| | |
|---|---|
| No | Sequence information |

| | | |
|---|---|---|
| 1. pFUSE-hIgG4-fc2-bsFlagellin- | DNA | SEQ ID NO: 38 |
| partial hinge (MSP303) | protein | SEQ ID NO: 39 |
| 2. pFUSE-hIgG4-fc2-bsFlagellin- | DNA | SEQ ID NO: 40 |
| partial hinge (MSP304) | protein | SEQ ID NO: 41 |
| 3. pFUSE-hIgG4-fc2-bsFlagellin- | DNA | SEQ ID NO: 42 |
| partial hinge-NL | protein | SEQ ID NO: 43 |
| 4. pFUSE-hIgG4-fc2-bsFlagellin- | DNA | SEQ ID NO: 44 |
| full hinge-CPSC (MSP305) | protein | SEQ ID NO: 45 |
| 5. pFUSE-hIgG4-fc2-bsFlagellin- | DNA | SEQ ID NO: 46 |
| full hinge-CPSC-NL | protein | SEQ ID NO: 47 |
| 6. pFUSE-hIgG4-fc2-bsFlagellin- | DNA | SEQ ID NO: 48 |
| full hinge-CPPC (MSP306) | protein | SEQ ID NO: 49 |
| 7. pFUSE-hIgG4-fc2-bsFlagellin- | DNA | SEQ ID NO: 50 |
| full hinge-CPPC-NL | protein | SEQ ID NO: 51 |
| 8. pFUSE-hIgG4-fc2-bsFlagellin- | DNA | SEQ ID NO: 52 |
| full hinge-CPPC-S220P | protein | SEQ ID NO: 53 |
| 9. pFUSE-hIgG4-fc2-bsFlagellin- | DNA | SEQ ID NO: 54 |
| full hinge-CPPC-G223T | protein | SEQ ID NO: 55 |
| 10. pFUSE-hIgG4-fc2-bsFlagellin- | DNA | SEQ ID NO: 56 |
| full hinge-CPPC-P224H | protein | SEQ ID NO: 57 |
| 11. pFUSE-hIgG4-fc2-bsFlagellin- | DNA | SEQ ID NO: 58 |
| full hinge-CPPC-P225T | protein | SEQ ID NO: 59 |
| 12. pFUSE-hIgG4-fc2-bsFlagellin- | DNA | SEQ ID NO: 60 |
| full hinge-CPPC (MSP306L) | protein | SEQ ID NO: 61 |

(2) Expression and Purification of Proteins from the Constructed Plasmids in Animal Cells
1) Coat the 12-well plate with 500 ul of poly-L-lysine per well and incubate for 5 minutes at room temperature (RT). Wash once with serum-free DMEM and once with 10% FBS DMEM. Seed HEK293T cells to achieve approximately 60% confluency on the day of transfection.
2) On the day of the experiment, mix 1 ug of DNA with 2 ul of TurboFect Transfection Reagent (Thermo Scientific) in 100 ul of serum-free DMEM and incubate for 20 minutes at RT. Transfect with a total volume of 1 ml.
3) 24 hours after transfection, change the media to 0.5% FBS DMEM.
4) Harvest 400 ul of media 24 hours after media change and use the supernatant after centrifugation at 12300 g for 1 minute to remove debris.
5) Alternatively, express the proteins in CHO S cell line using flask culture (200 ml media) with 6×10\hat{ }6 cells/ml and DNA at a concentration of 1 mg/L.
6) After culturing, purify each protein using affinity chromatography and size exclusion chromatography.

### (3) TLR5 Agonist Assay

The TLR5 activation ability of the (i) to (vi) proteins prepared according to the above method was confirmed using the following steps:
1) The proteins are quantified and prepared.
2) HEK-Blue cells are cultured in DMEM (4.5 g/L glucose, 2 mM L-glutamine), 10% FBS, 100 U/ml penicillin, 100 ug/ml streptomycin, and 100 ug/ml Normocin-containing media in a 75 T-Flask.
3) Thawed cells are cultured and subcultured in media containing Normocin (100 ug/ml) 3 times before being subcultured at least 2 times in media containing Normocin (100 ug/ml), Blasticidin (15 ug/ml), and Zeocin (100 ug/ml) before use in the experiment.
4) When the cell density in the Flask reaches 70˜80%, the media is removed, and the cells are detached by tapping and centrifuged (1000 rpm/5 min). The cells are then subcultured at a 1:6 ratio.
5) The cells are thawed, mixed well, and stored in a 4° C. refrigerator after 30 minutes of reaction at 37° C., wrapped in foil. Before use, they are warmed up to 37° C.
6) The protein or substance to be diluted is prepared according to the required concentration (up to 20 ul). The substance is added to the 96-well plate and placed on ice. PBS is added to the Blank well at 20 ul.
7) The cells are counted based on 140,000 cells/ml, mixed well with HEK-Blue detection solution, and then 180 ul is added to each well.
8) The cells are counted with a small amount of PBS, and the total amount is determined based on 96-well triplicates.
9) The plate is read at 620 nm after 16 hours of incubation at 37° C./5% CO2.

### (4) Biolayer Interferometry Analysis

### [TLR5avitag Biotinylation]

1) Sample preparation: BirA500: BirA biotin-protein ligase standard reaction kit was used for biotinylation.
2) TLR5avitag (˜1 mg/ml) was mixed with BiomixA (50 ul), BiomixB (50 ul), BirA (10 ul), and buffer (20 mM Hepes pH 7.4, 150 mM NaCl) to a final volume of 500 ul.
3) Incubation was carried out for 5 hours at 18° C.
4) Dialysis was performed three times against 1 L of buffer (20 mM Hepes pH 7.4, 150 mM NaCl) to remove residual substances.
5) Biotinylation was confirmed by SDS-PAGE gel loading, PVDF membrane transfer, and streptavidin-HRP detection.

### [BLI Experiment]

6) SA biosensors were hydrated in 200 μl of baseline buffer for at least 5 minutes.
7) Each sample protein was diluted in baseline buffer. TLR5avitag was diluted to 5 g/ml, and the ligand proteins (CPPC, entolimod, flagellin) were diluted to concentrations of 81, 27, 9, 3 nM, respectively.

### [Palate Measurement]

8) Set up the process in the Octet Data Acquisition program.
9) Plate definition: Input the type of plate well to be measured (refer to plate composition).
10) Assay Definition: Set the well and each step in order of lanes 1-9 for the sensor to move (refer to plate composition and settings for each step) and conduct the experiment.
11-1) Conduct sensor wash across lanes 1-3.
11-2) Fix TLR5avitag on the sensor in lane 4. 11-3) Conduct sensor wash across lanes 5-7.
11-4) Confirm the association between TLR5 and the ligand in lane 8.
11-5) Confirm the dissociation between TLR5 and the ligand in lane 9.
12) Verify that the baseline for each of the five sensors is properly established when the measurement begins.
13) Analyze the results obtained using the Octet Data Analysis program (Association and dissociation, setting for 1:1 binding model fitting).

### (5) Gel Filtration Experiment

### [Buffer & Column Conditions]

1) Buffer & Column conditions
   Gel-filtration buffer: 20 mM HEPES (Biobasic, HB0264) pH 7.4, 150 mM NaCl (Biobasic, DB0483)
   All proteins were in gel-filtration buffer conditions and protein binding between proteins was also performed under gel-filtration buffer conditions. The column environment was also prepared with gel-filtration buffer for the experiment.
   Column: Superdex 200 10/300 GL
   FPLC: AKTA FPLC (GE-healthcare)
2) Each protein was individually injected into the column to determine their molecular weight and elution position.

### [Sample Mixing & Chromatography Running]

3) The concentrations of each protein were calculated, and TLR5 and ligand protein were mixed in ratios of 1:0, 1:1, 1:2, and 0:1, respectively. The total volume was adjusted to 300 μl using gel-filtration buffer.
4) Equilibration was carried out by passing more than 3 column volumes (75 ml) of gel-filtration buffer through the column.
5) Equilibration was performed at a flow rate of 0.5 ml/min for 1 ml.
6) After incubating for 30 minutes at 18° C., the sample was centrifuged (12000 rpm, 4° C., min) to sediment the precipitate, and the supernatant was collected for injection into the FPLC. (The sample was injected into the sample loop using a syringe.)
7) The elution peak of the sample was checked by passing 1 column volume (25 ml) of gel-filtration buffer and measuring the UV 280 nm results.

### 2. Experimental Results

### (1) TLR5 Agonist Assay

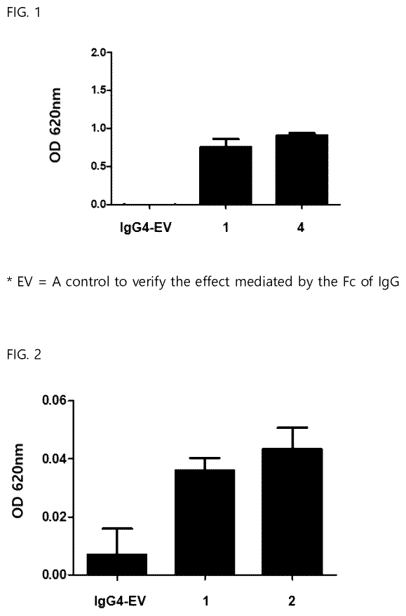

The TLR5 activation ability of each protein prepared according to the experimental method was evaluated for fusion proteins 1 to 11, and the results are shown in FIGS. 1 to 5.

As shown in FIG. 1, both the protein with a partial hinge sequence of hIgG4 Fc fused (fusion protein 1) and the protein with the entire hinge sequence fused (fusion protein 4) exhibited TLR5 activation ability, with a higher degree of activation observed in the protein with the entire hinge sequence fused.

As shown in FIG. 2 both the fusion protein with one linker (GGGGS) (SEQ ID NO: 12) connecting Bsflagellin and hIgG4 Fc (fusion protein 1) and the fusion protein (fusion protein 2) with three linkers (GGGGS) (SEQ ID NO: 13) exhibited TLR5 activation ability, with a higher degree of activation observed in the fusion protein with three linkers.

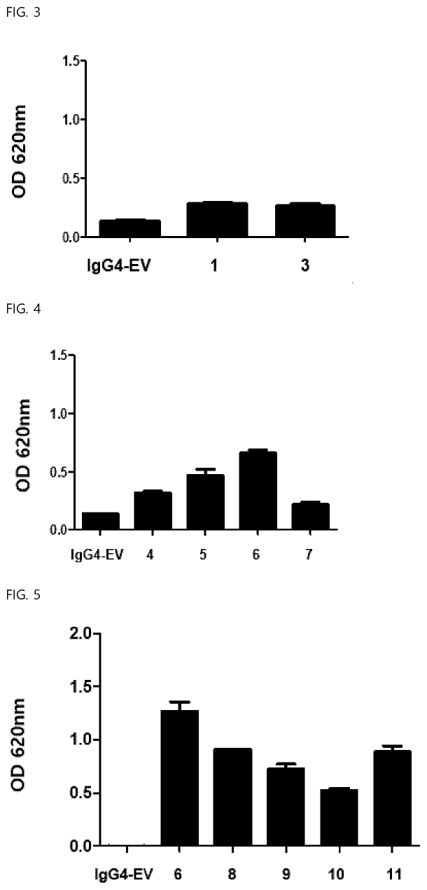

As shown in FIG. 3, both the fusion protein with one linker (GGGGS) (SEQ ID N; 12) connecting Bsflagellin and hIgG4 Fc (fusion protein 1) and the fusion protein without a linker (fusion protein 3) exhibited TLR5 activation ability, with similar degrees of activation observed.

As shown in FIG. 4, it was confirmed that the TLR5 activation ability of the fusion protein 6 fused with hIgG4 Fc containing a mutation (S228P) that prevents Fab arm exchange was significantly higher than that of the fusion protein 4 fused with wild-type hIgG4 Fc.

As shown in FIG. 5, it was confirmed that both the fusion proteins including hIgG4 Fc with the Fab-arm exchange preventing mutation (S228P) (fusion protein 6) and charge variants containing additional mutations exhibited TLR5 activation ability.

### (2) Analysis of Binding Affinity Between Fusion Protein and TLR5

Protein-protein interactions between the fusion protein 6 (MSP306) and zTLR5 were analyzed by biolayer interferometry for fusion protein-TLR5 binding, and complex formation between the fusion protein and TLR5 was analyzed by gel-filtration chromatography.

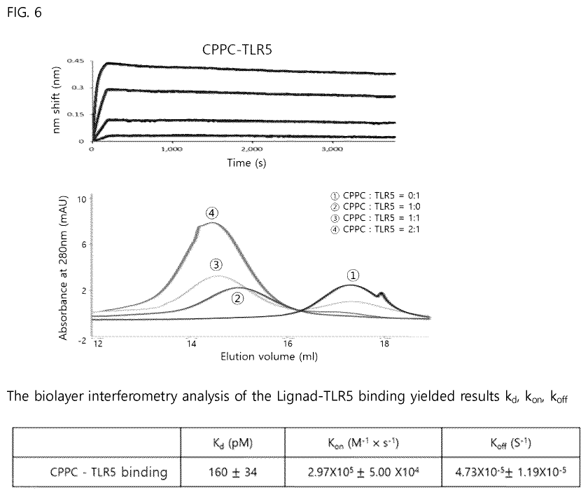

The results are presented in FIG. 6.

As shown in FIG. 6, the fusion protein 6 exhibited a very high binding affinity at the pM level.

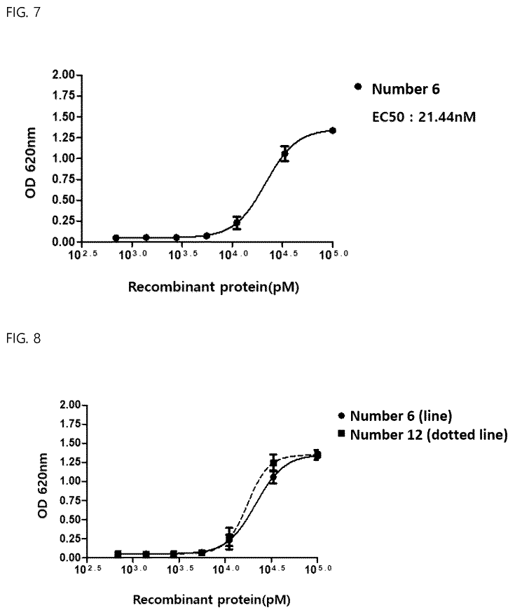

In addition, the EC50 value of TLR5 activation by the fusion protein 6 was analyzed and evaluated, as shown in FIG. 7, with an EC50 of 21.44 nM.

In addition, when comparing the TLR5 activation potency of the most effective fusion protein 6 with that of the fusion protein 12, which differs from the fusion protein 6 only in the 3rd sequence of the C-terminus of hIgG4 Fc, as shown in FIG. 8, it was confirmed that the activity of the fusion protein 6 and 12 was almost the same.

### 3. Protein Expression and Nasal Absorption Experiments in Insect Cells

### (1) Protein Expression in Insect Cells

### [Production of Original Baculovirus]

1) Seed 100×10\hat{ }4 Sf9 cells per well in a 6-well plate.
2) Fill up to 2 ml with antibiotic-free culture medium and incubate at 28° C. for 30 minutes. Proceed with the next step during incubation.
3) Mix 8 μl of CellfectinII solution (gibco) with 100 μl of antibiotic-free culture medium and incubate at room temperature for 30 minutes.
4) In a separate container, add 1.5 μl of midi-prep DNA (SEQ ID NO: 62, concentration of 1 μg/μl) and 1.5 μl of linearized baculovirus DNA, mix gently, and incubate at room temperature for 5 minutes. Add 100 μl of antibiotic-free culture medium and mix well.
5) Mix the solution from step 3) and the solution from step 4), and incubate at room temperature for 30 minutes.
6) Remove the plate from step 2), discard the old culture medium, and add 1 ml of fresh culture medium to each well.
7) Add the solution from step 5) to each well, seal the plate, and incubate at low temperature for 4 hours.
8) Wash with culture medium and replace with antibiotic-free culture medium.
9) Seal the plate and incubate at 28° C. for 5 days.
10) If storing the Original Baculovirus for later use instead of making Primary Baculovirus right away, collect the culture medium and store at 4° C. Follow the next steps for immediate use.

### [Production of Primary Baculovirus]

1) Cultivate Sf9 cells to the logarithmic phase before starting the experiment.
2) Seed 1000×10\hat{ }4 cells of Sf9 cells into a 75 T flask and let them settle for 30 minutes at 28° C.
3) Confirm that Sf cells have attached to the bottom of the flask, then remove the culture medium.
4) Add 1 ml of fresh culture medium and then drop 500 ul of original baculovirus using a pipette. Gently shake to ensure even distribution of the virus and incubate at 28° C. for 1 hour.
5) Add 10 ml of fresh culture medium and incubate at 28° C. for 3 days.
6) If the Primary Baculovirus is not used to immediately make Secondary Baculovirus, collect the culture medium and store at 4° C. If making the Secondary Baculovirus right away, follow the next steps.

### [Production of Secondary Baculovirus]

1) Prior to the experiment, culture Sf9 cells to the log phase.
2) Seed 2000×10\hat{ }4 Sf9 cells in a 175 T flask and incubate at 28° C. for 30 minutes.
3) After confirming that the Sf cells have adhered to the flask bottom, remove all the culture medium.
4) Add 2 ml of fresh culture medium, then drop 500 ul of primary baculovirus using a pipette. Gently shake the flask to ensure that the primary baculovirus is distributed well and incubate for 1 hour at 28° C.
5) Add 20 ml of fresh culture medium and incubate at 28° C. for 3 days.
6) If not producing Tertiary Baculovirus immediately with Secondary Baculovirus, collect the culture medium and store it at 4° C. If producing immediately, follow the next steps

### [Production of Tertiary Baculovirus]

Once protein expression is confirmed, Tertiary Baculovirus is produced using a shaker incubator.
1) Prior to the experiment, Sf9 cells are cultured to reach log phase.
2) 180-200×10\hat{ }4 Sf9 cells are seeded in a flask, followed by the addition of an appropriate amount of Secondary Baculovirus. The culture is then incubated at 28° C. with shaking at 90 rpm for 3 days.
3) The culture is collected and stored at 4° C.

### [Protein Purification]

1) Seed Hi5 cells at a density of 60-80×10\hat{ }4cells/ml.
2) Cultivate Hi5 cells until a density of 180-200×10\hat{ }4cells/ml is reached. Add an appropriate amount of Tertiary Baculovirus and culture for 3 days at 28° C. and 90 rpm in a shaking incubator.
3) Collect Hi5 cells in a tube and centrifuge at 4° C. and 8000 rpm for 10 minutes.
4) While step 3) is being performed, add 5 ml of Roche Ni resin to a column and pass 10 ml of [20 mM HEPES (pH 7.0)+200 mM NaCl+30 mM Imidazole] solution through it.
5) After the centrifugation of step 3) is complete, collect the supernatant and pass it through the resin prepared in step 4).
6) Pass 150 ml of [20 mM HEPES (pH 7.0)+200 mM NaCl+30 mM Imidazole] solution through the resin.
7) Once protein is confirmed on the resin, pass the following three types of solutions, each with a volume of 15 ml:
   Solution 1: 20 mM HEPES (pH 7.0)+200 mM NaCl+100 mM Imidazole
   Solution 2:20 HEPES (pH 7.0)+200 mM NaCl+300 mM Imidazole
   Solution 3: 20 mM HEPES (pH 7.0)+200 mM NaCl+500 mM Imidazole
8) After confirming the protein using SDS-PAGE, concentrate it.
9) Dilute Imidazole to 1/10 by adding 9 ml of 20 mM HEPES (pH 7.0)+200 mM NaCl solution and concentrate again.
10) Purified protein with (SEQ ID NO: 63) was obtained through SEC by FPLC and confirmed using peak analysis and SDS-PAGE (20 mM HEPES (pH 7.0)+200 mM NaCl).

### (2) In Vivo Imaging of Intranasally Administered Substances

In vivo imaging of Bsflagellin and the fusion protein with (SEQ ID NO: 63) expressed in insect cells was performed over time according to the following steps:

### [Experimental Preparation]

1) Male SKH1 mice at 8 weeks of age were purchased and acclimatized before use in the experiments.
2) BsFlagellin, the comparator to the fusion protein with (SEQ ID NO: 63), was purchased from Invivogen and used in the experiments.

### [Labeling of Substances]

Labeling of substances for in vivo tracking was performed according to the following steps:
1) Flamma 774 NHS ester (pws1603, bioacts) was diluted to 1 mg.
2) BsFlagellin or the fusion protein with (SEQ ID NO: 63) was mixed with Flamma 774 NHS ester.
3) The mixture was incubated at 4° C. for 3 days.
4) After the reaction, unbound material was separated using an Amicon Ultra centrifugal filter unit.
5) The separated material was quantified using the BCA assay to determine the concentration.

### [In Vivo Imaging Analysis]

1) The prepared substance from the previous stage was injected into SKH1 mice (n=2) via the nasal cavity at a dose of 10 ug/head.
2) PBS was injected into SKH1 mice (n=1) at the same volume.
3) The mice were anesthetized by inhalation for in vivo imaging analysis.
4) The anesthetized mice were injected via the nasal cavity, and fluorescence images were obtained up to the neck region of the mice using the IVIS™ imaging system (Perkin Elmer) at 3, 6, and 24 hours after injection.

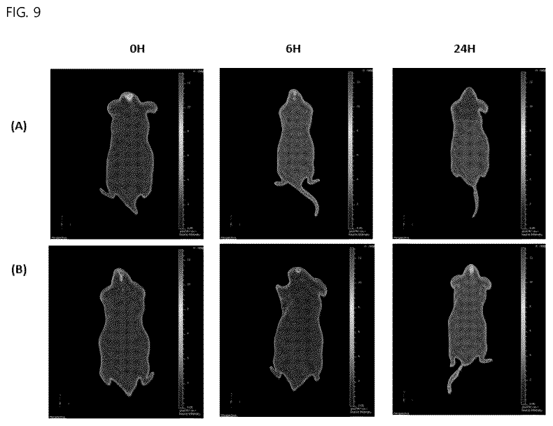

The results are shown in FIG. 9.

As shown in FIG. 9, fluorescence labeling of both substances was detected in the olfactory epithelium and lymph nodes (deep cervical, mandibular) of all mice when Bsflagellin (FIG. 9(B)) or fusion protein with (SEQ ID NO: 63) (FIG. 9(A)) was administered via nasal route.

In the fluorescence imaging at 24 hours after substance injection, it was confirmed that the fusion protein with (SEQ ID NO: 63) was absorbed more rapidly in the body than Bsflagellin (B).

### INDUSTRIAL APPLICABILITY

The fusion protein provided by the present invention not only exhibits significantly superior Toll-like receptor 5 (TLR5) pathway activation compared to wild-type flagellin, fragment, or variant thereof, but also exhibits significantly superior TLR5 pathway activation compared to a protein fused with wild-type IgG4 Fc and flagellin. Therefore, it can be very useful for the development of therapeutic agents and/or vaccine adjuvants for diseases that can be prevented, improved, or treated through TLR5 pathway activation. Thus, there is a high potential for industrial applicability.

1. A fusion protein comprising flagellin, fragment, or variant thereof, and a human IgG4 Fc variant, wherein the human IgG4 Fc variant has a mutation that prevents Fab arm exchange.

2. The fusion protein according to claim 1, wherein the flagellin is derived from a microorganism selected from the group consisting of Bacillus, Salmonella, Helicobacter, Vibrio, Serratia, Shigella, Treponema, Legionella, Borrelia, Clostridium, Agrobacterium, Bartonella, Proteus, Pseudomonas, Escherichia, Listeria, Yersinia, Campylobacter, Roseburia, and Marinobacter.

3. The fusion protein according to claim 1, wherein the flagellin is derived from a microorganism selected from a group consisting of Salmonella enteritidis, Salmonella typhimurium, Salmonella Dublin, Salmonella enterica, Helicobacter pylori, Vibrio cholera, Vibrio vulnificus, Vibrio fibrisolvens, Serratia marcesens, Shigella flexneri, Treponema pallidum, Borrelia burgdorferei, Clostridium difficile, Agrobacterium tumefaciens, Bartonella clarridgeiae, Proteus mirabilis, Bacillus subtilis, Bacillus cereus, Bacillus halodurans, Pseudomonas aeruginosa, Escherichia coli, Listeria monocytogenes, Yersinia pestis, Campylobacter spp, Roseburia spp, and Marinobacter spp.

4. The fusion protein according to claim 1, wherein the flagellin comprises a conserved sequence which is recognized by TLR5 (toll-like receptor 5).

5. The fusion protein according to claim 1, wherein the fragment has a hypervariable region removed from wild-type flagellin.

6. The fusion protein according to claim 1, wherein the fragment comprises at least one domain selected from the group consisting of C-terminal domain 0, C-terminal domain 1, C-terminal domain 2, N-terminal domain 2, N-terminal domain 1, N-terminal domain 0 of wild type flagellin, and a domain having 80% or greater amino acid sequence homology with the said domain.

7. The fusion protein according to claim 1, wherein the variant has 80% or greater amino acid sequence homology with wild-type flagellin and exhibits Toll-like receptor 5 (TLR5) stimulatory activity.

8. The fusion protein according to claim 1, wherein the human IgG4 Fc variant comprises a hinge.

9. The fusion protein according to claim 1, wherein the hinge consists of 4 to 12 amino acids comprising CPPC sequence.

10. The fusion protein according to claim 1, wherein the mutation that prevents Fab arm exchange is a mutation which imparts inter-heavy chain disulfide bond formation of the IgG4 Fc.

11. The fusion protein according to claim 1, wherein the human IgG4 Fc variant comprises at least one amino acid substitution selected from the group consisting of a substitution of Ser with Pro at position 228 (S228P), a substitution of Arg with Lys at position 409 (R409K), or a combination thereof.

12. The fusion protein, wherein the human IgG4 Fc variant further comprises one or more amino acid substitutions selected from a group consisting of S220P, G223T, P224H, and P225T at positions 220, 223, 224, and 225 of the wild-type human IgG4 Fc, respectively.

13. The fusion protein according to claim 1, wherein the human IgG4 Fc variant comprises a substitution of Leu with Pro at the third amino acid from the C-terminus of wild-type human IgG4 Fc.

14. The fusion protein according to claim 1, wherein the N-terminus or C-terminus of the flagellin, a fragment thereof or a variant thereof is linked to the N-terminus or C-terminus of the human IgG4 Fc variant.

15. The fusion protein according to claim 1, wherein the flagellin, fragment, or variant thereof is directly connected or connected via a linker to the human IgG4 Fc variant.

16. The fusion protein according to claim 1, wherein the fragment or the variant thereof consists of an amino acid sequence selected from the group consisting of SEQ ID NO 1 to 5 or an amino acid sequence showing 80% or more sequence homology thereto.

17. The fusion protein according to claim 1, wherein the human IgG4 Fc variant comprises an amino acid sequence selected from the group consisting of SEQ ID NO: 6 to SEQ ID NO: 11.

18. The fusion protein according to claim 15, wherein the linker consists of an amino acid sequence of (GGGGS) n (SEQ ID NO: 12, n is 1 to 5).

19. The fusion protein according to claim 15, wherein the fusion protein comprises an amino acid sequence of SEQ ID NO: 49, SEQ ID NO: 51, SEQ ID NO: 53, SEQ ID NO: 55, SEQ ID NO: 57, SEQ ID NO: 59, SEQ ID NO: 61, or SEQ ID NO: 63.

20. A polynucleotide encoding the fusion protein of any one of claims 1 to 19.

21. A vector comprising the polynucleotide of claim 20.

22. A transfectant transformed with the vector of claim 21.

23. A pharmaceutical composition comprising the fusion protein of any one of claims 1 to 19 as an active component.

24. The pharmaceutical composition according to claim 23, wherein The pharmaceutical composition exhibits Toll-like receptor 5 (TLR5) stimulation activity.

25. The pharmaceutical composition according to claim 23, wherein The pharmaceutical composition is effective for the prevention or treatment of radiation exposure-induced damage, reperfusion injury, inflammatory bowel disease, autoimmune disease, viral infection, metabolic disease or cancer, or the enhancement of immune function.

26. The pharmaceutical composition according to claim 23, wherein the radiation exposure-induced damage is gastrointestinal syndrome or hematopoietic syndrome.

27. The pharmaceutical composition according to claim 23, wherein the aging is at least one selected from alopecia, cataract, hernia, colitis, osteoporosis and osteomalacia caused by aging.

28. A vaccine adjuvant comprising the fusion protein of any one of claims 1 to 19 as an active ingredient.

29. Use of the fusion protein of any one of claims 1 to 19 for the preparation of an agent for treating radiation exposure-induced damage, reperfusion injury, inflammatory bowel disease, autoimmune disease, viral infection, metabolic disease or cancer, or enhancing immune function.

30. A method for treating radiation exposure-induced damage, reperfusion injury, inflammatory bowel disease, autoimmune disease, viral infection, metabolic disease or cancer, or enhancing immune function, the method comprising administering an effective amount of a composition comprising the fusion protein of any one of claim 1 as an active ingredient to a subject in need thereof.
